(19) United States
(12) Patent Application Publication
(10) Pub. No.: US 2006/0177823 A1
(43) Pub. Date: Aug. 10, 2006

(54) Molecules

(75) Inventor: Niklas Dahl, Uppsala (SE)

Correspondence Address: FISH & NEAVE IP GROUP;ROPES & GRAY LLP, ONE INTERNATIONAL PLACE, BOSTON, MA, 02110-2624, US

(73) Assignee: AstraZeneca AB, Sodertalje (SE)

(21) Appl. No.: 10/549,460

(22) PCT Filed: Mar. 16, 2004
(86) PCT No.: PCT/GB04/01124

§ 371 Date: Sep. 16, 2005

(30) Foreign Application Priority Data
Mar. 19, 2003 (GB) 0306185.0

(51) Int. Cl. C12Q 1/68; C07H 21/04; C12P 21/06; C07K 14/47

(52) U.S. Cl. 435/6; 435/69.1; 435/320.1; 435/325; 530/350; 536/23.5

(57) ABSTRACT

The present invention describes the chromosomal location of obesity susceptibility genes and provides methods for detecting the presence of such genes. The present invention enables the development of novel therapies to treat obesity.

The present invention describes the chromosomal location of one or more genes involved in obesity.

In recent years, there has been an increase in the population of patients with what are termed “life-style related” diseases, such as obesity and hyperlipidemia. Obesity refers to a physical state in which the stored fat, constituted mainly of triglycerides, is accumulated excessively in the body. A consequence of obesity is an increased risk of arteriosclerosis, fatty liver, hypertension, diabetes, coronary heart disease, stroke, gallbladder disease, osteoarthritis, respiratory problems and some types of cancer (Visscher & Seidell, Annu Rev Public Health (2001); 22(1): 355-75). It has been shown recently that there can also exist a strong genetic component underlying the development of obesity. The role of certain genes, e.g., agouti, leptin (ob) and leptin receptor (db), in the regulation of body fat distribution has been described (Friedman & Halaas, Nature (October 1998); 395(6704): 763-70; Ahima et al, Front Neuroendocrinol (July 2000); 21(3): 263-307). In humans, mutations in leptin, leptin receptor, melanocortin 4-receptor (MC4-R) and peroxisome proliferator-activated receptor (PPAR) γ2 genes have been shown to be important component in patients with severe obesity (Clement et. al.; Int J Obes Relat Metab Disord 2000 March; 24(3): 391-3, Yeo et. al.; Nat Genet 1998 October; 20(2): 111-2).

The identification of other genes involved in the development of obesity will provide a better understanding of the disease, and an opportunity to develop more specific and effective drugs.

The present invention is based on the discovery of a chromosomal translocation which has a predisposing effect on the development of obesity. The translocation exists between chromosome 4 and chromosome 15. Specifically, chromosome 4 has been broken at cytoband 4q22.3 and chromosome 15 has been broken at cytoband 15q22.2. The outcome of the translocation event is that at least two genes are affected in persons having this translocation and that persons having this translocation are more susceptible to develop obesity.

Two of the genes affected by the translocation are uncoordinated 5C (UNC5C) residing on chromosome 4 and RAR-like orphan receptor alpha 1 (RORα1) residing on chromosome 15.

The breakpoint at chromosome 4 occurs between the genes pyruvate dehydrogenase A2 (PDHA2) and UNC5C. While neither of these genes are destroyed in the mutation event, UNC5C transcription is upregulated in adipocytes. A cDNA encoding UNC5C is set out as SEQ ID NO: 9 (with the first nucleotide of the UNC5C coding region accorded position 155), and its gene product is set out as SEQ ID NO: 10.

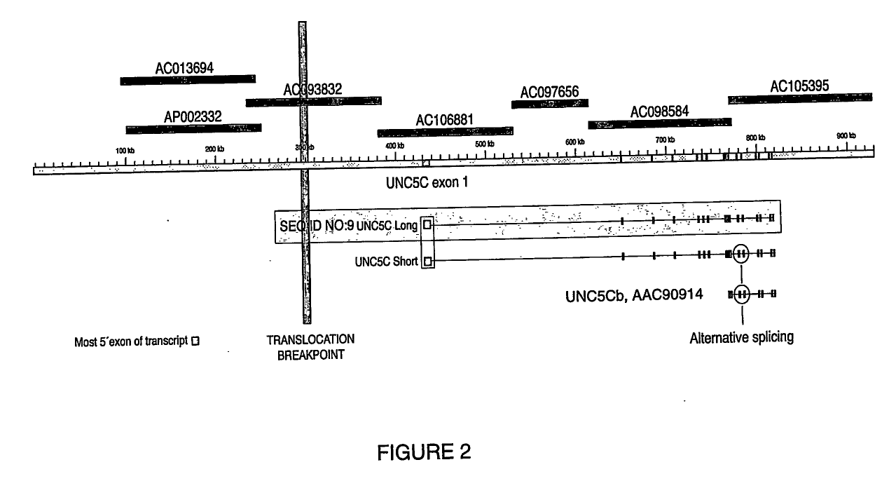

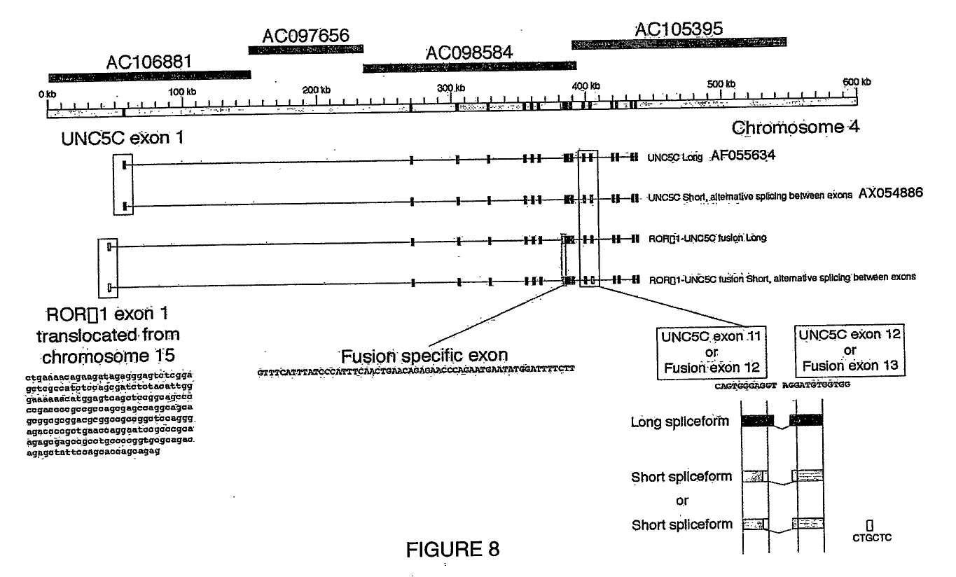

There is an alternative splice version produced from the UNC5C gene, where the exons 11 and 12 are spliced together from internal sites producing a transcript with a truncated open reading frame and a much shorter protein. A sequence containing this splice event has been determined and submitted to the GENESEQN database and has accession number AAC90914 (FIG. 2 and FIG. 8). We confirmed the presence of this alternative transcript with the use of PCR on human brain material. A putative “in silico” generated full length clone of the alternative transcript is set out as SEQ ID NO: 13 (with the first nucleotide of the sUNC5C coding region accorded position 1) and its gene product is set out as SEQ ID NO 14. As used herein “sUNC5C” refers to this shorter splice version of UNC5C unless otherwise stated. The sequence AAC90914 is a truncated version of sUNC5C.

The breakpoint at chromosome 15 is within the first intron of RORα1 and this mutation event destroys the transcription of the RORα1 isoform. A cDNA encoding RORα1 is set out as SEQ ID NO: 5 (with the first nucleotide of the RORα1 coding region accorded position 102), and its gene product is set out as SEQ ID NO:6. The translocation results in the first exon of RORα1 being spliced into the second exon of UNC5C, resulting in the expression of two novel transcripts which are fusions between RORα1 and UNC5C (long and short). These two novel transcripts are expressed in affected patients adipocytes. The first 657 amino acids are identical for both fusion proteins.

The first transcript results in the translation of a long fusion protein between RORα1-UNC5C (termed herein “1RORα1-UNC5C”). A cDNA encoding 1RORα1-UNC5C is set out as SEQ ID NO: 1, and its protein fusion product as SEQ ID NO:2. In SEQ ID NO: 1, nucleotide positions 1-233 represent that part of the transcript from RORα1. Nucleotide positions 234-2986 represent that part of the transcript from UNC5C. In SEQ ID NO: 2, amino acid positions 1-55 represent that part of the protein fusion product derived from RORα1 DNA. Amino acid positions 56-383 and 403-964 represent that part of the protein fusion product derived from UNC5C DNA. The 1RORα1-UNC5C transcript is produced by splicing RORα1 exon1 with UNC5C exon 2-16 together with a fusion specific exon situated between UNC5C exons 7 and 8. The fusion specific exon is an extra exon of 57 bp which encodes for an extra 19 amino acid residues and resides within the IRORα1-UNC5C transcript at nucleotide positions 1220-1276 of SEQ ID NO:1. In SEQ ID NO:2, amino acid positions 384-402 represent that part of the protein fusion product derived from the fusion specific exon. It is to be noted that the first coding nucleotide for the amino acid position 384 comes from UNC5C exon 7.

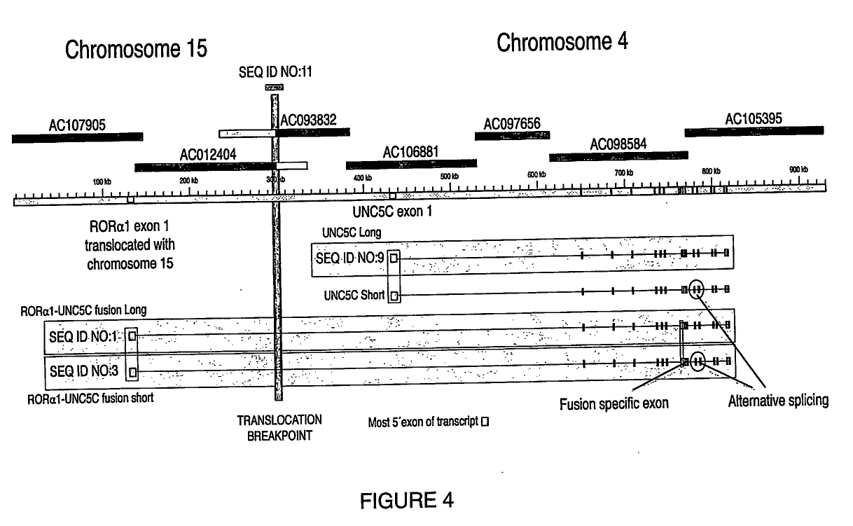

The second transcript results in the translation of a shorter fusion protein between RORα1-UNC5C (known herein as the “sRORα1-UNC5C”). A cDNA encoding sRORα1-UNC5C is set out as SEQ ID NO: 3, and its protein fusion product as SEQ ID NO: 4. In SEQ ID NO: 3, nucleotide positions 1-233 represent that part of the transcript from RORα1. Nucleotide positions 234-2546 represent that part of the transcript from UNC5C. In SEQ ID NO:4, amino acid positions 1-55 represent that part of the protein fusion product derived from RORα1 DNA. Amino acid positions 56-383 and 403-669 represent that part of the protein fusion product derived from sUNC5C DNA. As for the long form, a fusion specific exon is situated between UNC5C exons 7 and 8. The fusion specific exon is an extra exon of 57 bp which encodes for an extra 19 amino acid residues and resides within the sRORα1-UNC5C transcript at nucleotide positions 1220-1276 of SEQ ID NO:3. In SEQ ID NO:4, amino acid positions 384-402 represent that part of the protein fusion product derived from the fusion specific exon. It is to be noted that the first coding nucleotide for the amino acid position 384 comes from UNC5C exon 7. The sRORα1-UNC5C transcript only differs from 1RORα1-UNC5C in the way that UNC5C exons 11 and 12 are spliced together. sRORα1-UNC5C uses internal splice sites within UNC5C exon 11 and 12 so that the 3′ part of exon 11 and the 5′ part of exon 12 is omitted (FIG. 4 and Tables 1 and 2). Thus, sRORα1-UNC5C has a destroyed open reading frame resulting in the deletion of 307 amino acids and replacing them with 12 amino acids from another reading frame.

Table 1 below shows DNA sequence alignment of the 1RORα1-UNC5C (SEQ ID NO: 1) and sRORα1-UNC5C (SEQ ID NO: 3) fusion transcripts together with UNC5C(AF055634) and the alternative spliceform of UNC5C (AAC90914). Also shown are the genomic exon sequences of RORα1 exon 1 and UNC5C exon 1-16:

TABLE 1

| | |
|---|---|
| | 1 42 |
| AF055634 | CTGCCTTTGGAGAAAGTGGAGTGTGGCGCTTGGTTGTCGTTAT |
| UNC5C_exon1 | CTGCCTTTGGAGAAAGTGGAGTGTGGCGCTTGGTTGTCGTTAT |
| | 43 102 |
| AF055634 | TTCTTCGGACTGCTTCGCGGTGCACGGATTCAGCTTCTGCCCAGTGGGGCTTTCAGCTGT |
| UNC5C_exon1 | TTCTTCGGACTGCTTCGCGGTGCACGGATTCAGCTTCTGCCCAGTGGGGCTTTCAGCTGT |
| SEQ ID NO:1 | CTGAAAACAGAAGATAGAGGGAGTCTCGGAGCTCGCCATCTCCAGCGATCTCTACATTG |
| SEQ ID NO:3 | CTGAAAACAGAAGATAGAGGGAGTCTCGGAGCTCGCCATCTCCAGCGATCTCTACATTG |
| RORA1_exon1 | CTGAAAACAGAAGATAGAGGGAGTCTCGGAGCTCGCCATCTCCAGCGATCTCTACATTG |
| | 103 162 |
| AF055634 | TTGCGCGTCTCTCTGTCCCCCTCCCCTCCCCCCGGCACACCTCTGTCTACGATGAGGAAA |
| UNC5C_exon1 | TTGCGCGTCTCTCTGTCCCCCTCCCCTCCCCCCGGCACACCTCTGTCTACGATGAGGAAA |
| SEQ ID NO:1 | GGAAAAAACATGGAGTCAGCTCCGGCAGCCCCCGACCCCGCCGCCAGCGAGCCAGGCAGC |
| SEQ ID NO:3 | GGAAAAAACATGGAGTCAGCTCCGGCAGCCCCCGACCCCGCCGCCAGCGAGCCAGGCAGC |
| RORA1_exon1 | GGAAAAAACATGGAGTCAGCTCCGGCAGCCCCCGACCCCGCCGCCAGCGAGCCAGGCAGC |
| | 163 222 |
| AF055634 | GGTCTGCGGGCGACAGCGGCCCGCTGCGGACTGGGACTGGGATACTTGCTGCAAATGCTC |
| UNC5C_exon1 | GGTCTGCGGGCGACAGCGGCCCGCTGCGGACTGGGACTGGGATACTTGCTGCAAATGCTC |
| SEQ ID NO:1 | AGCGGCGCGGACGCGGCCGCCGGCTCCAGGGAGACCCCGCTGAACCAGGAATCCGCCCGC |
| SEQ ID NO:3 | AGCGGCGCGGACGCGGCCGCCGGCTCCAGGGAGACCCCGCTGAACCAGGAATCCGCCCGC |
| RORA1_exon1 | AGCGGCGCGGACGCGGCCGCCGGCTCCAGGGAGACCCCGCTGAACCAGGAATCCGCCCGC |
| | 223 282 |
| AF055634 | GTGCTACCTGCCCTGGCCCTGCTCAGCGCCAGCGGCACTGGCTCCGCCGCCCAAGATGAT |
| UNC5C_exon1 | GTGCTACCTGCCCTGGCCCTGCTCAGCGCCAGCGGCACTGGCTCCGCCGCCCAAG |
| UNC5C_exon2 | ATGAT |
| SEQ ID NO:1 | AAGAGCGAGCCGCCTGCCCCGGTGCGCAGACAGAGCTATTCCAGCACCAGCAGAGATGAT |
| SEQ ID NO:3 | AAGAGCGAGCCGCCTGCCCCGGTGCGCAGACAGAGCTATTCCAGCACCAGCAGAGATGAT |
| RORA1_exon1 | AAGAGCGAGCCGCCTGCCCCGGTGCGCAGACAGAGCTATTCCAGCACCAGCAGAG |
| | ***** |
| | 283 342 |
| AF055634 | GACTTTTTTCATGAACTCCCAGAAACTTTTCCTTCTGATCCACCTGAGCCTCTGCCACAT |
| UNC5C_exon2 | GACTTTTTTCATGAACTCCCAGAAACTTTTCCTTCTGATCCACCTGAGCCTCTGCCACAT |
| SEQ ID NO:1 | GACTTTTTTCATGAACTCCCAGAAACTTTTCCTTCTGATCCACCTGAGCCTCTGCCACAT |
| SEQ ID NO:3 | GACTTTTTTCATGAACTCCCAGAAACTTTTCCTTCTGATCCACCTGAGCCTCTGCCACAT |
| | ************************************************************ |
| | 343 402 |
| AF055634 | TTCCTTATTGAGCCTGAAGAAGCTTATATTGTGAAGAATAAGCCTGTGAACCTGTACTGT |
| UNC5C_exon2 | TTCCTTATTGAGCCTGAAGAAGCTTATATTGTGAAGAATAAGCCTGTGAACCTGTACTGT |
| SEQ ID NO:1 | TTCCTTATTGAGCCTGAAGAAGCTTATATTGTGAAGAATAAGCCTGTGAACCTGTACTGT |
| SEQ ID NO:3 | TTCCTTATTGAGCCTGAAGAAGCTTATATTGTGAAGAATAAGCCTGTGAACCTGTACTGT |
| | ************************************************************ |
| | 403 462 |
| AF055634 | AAAGCAAGCCCTGCCACCCAGATCTATTTCAAGTGTAATAGTGAATGGGTTCATCAGAAG |
| UNC5C_exon2 | AAAGCAAGCCCTGCCACCCAGATCTATTTCAAGTGTAATAGTGAATGGGTTCATCAGAAG |
| SEQ ID NO:1 | AAAGCAAGCCCTGCCACCCAGATCTATTTCAAGTGTAATAGTGAATGGGTTCATCAGAAG |
| SEQ ID NO:3 | AAAGCAAGCCCTGCCACCCAGATCTATTTCAAGTGTAATAGTGAATGGGTTCATCAGAAG |
| | ************************************************************ |
| | 463 522 |
| AF055634 | GACCACATAGTAGATGAAAGAGTAGATGAAACTTCCGGTCTCATTGTCCGGGAAGTGAGC |
| UNC5C_exon2 | GACCACATAGTAGATGAAAGAGTAGATGAAACTTCCG |
| UNC5C_exon3 | GTCTCATTGTCCGGGAAGTGAGC |
| SEQ ID NO:1 | GACCACATAGTAGATGAAAGAGTAGATGAAACTTCCGGTCTCATTGTCCGGGAAGTGAGC |
| SEQ ID NO:3 | GACCACATAGTAGATGAAAGAGTAGATGAAACTTCCGGTCTCATTGTCCGGGAAGTGAGC |
| | ************************************************************ |
| | 523 582 |
| AF055634 | ATTGAGATTTCGCGCCAGCAAGTGGAAGAACTCTTTGGACCTGAAGATTACTGGTGCCAG |
| UNC5C_exon3 | ATTGAGATTTCGCGCCAGCAAGTGGAAGAACTCTTTGGACCTGAAGATTACTGGTGCCAG |
| SEQ ID NO:1 | ATTGAGATTTCGCGCCAGCAAGTGGAAGAACTCTTTGGACCTGAAGATTACTGGTGCCAG |
| SEQ ID NO:3 | ATTGAGATTTCGCGCCAGCAAGTGGAAGAACTCTTTGGACCTGAAGATTACTGGTGCCAG |
| | ************************************************************ |
| | 583 642 |
| AF055634 | TGTGTGGCCTGGAGCTCCGCGGGTACCACAAAGAGCCGGAAGGCGTATGTGCGCATTGCA |
| UNC5C_exon3 | TGTGTGGCCTGGAGCTCCGCGGGTACCACAAAGAGCCGGAAGGCGTATGTGCGCATTGCA |
| SEQ ID NO: 1 | TGTGTGGCCTGGAGCTCCGCGGGTACCACAAAGAGCCGGAAGGCGTATGTGCGCATTGCA |
| SEQ ID NO: 3 | TGTGTGGCCTGGAGCTCCGCGGGTACCACAAAGAGCCGGAAGGCGTATGTGCGCATTGCA |
| | ************************************************************ |
| | 643 702 |
| AF055634 | TATCTACGGAAGACATTTGAGCAGGAACCCCTAGGAAAGGAAGTGTCTTTGGAACAGGAA |
| UNC5C_exon3 | T |
| UNC5C_exon4 | ATCTACGGAAGACATTTGAGCAGGAACCCCTAGGAAAGGAAGTGTCTTTGGAACAGGAA |
| SEQ ID NO:1 | TATCTACGGAAGACATTTGAGCAGGAACCCCTAGGAAAGGAAGTGTCTTTGGAACAGGAA |
| SEQ ID NO:3 | TATCTACGGAAGACATTTGAGCAGGAACCCCTAGGAAAGGAAGTGTCTTTGGAACAGGAA |
| | ************************************************************ |
| | 703 762 |
| AF055634 | GTCTTACTCCAGTGTCGACCACCTGAAGGGATCCCAGTGGCTGAGGTGGAATGGTTGAAA |
| UNC5C_exon4 | GTCTTACTCCAGTGTCGACCACCTGAAGGGATCCCAGTGGCTGAG |
| UNC5C_exon1 | GTGGAATGGTTGAAA |
| SEQ ID NO:1 | GTCTTACTCCAGTGTCGACCACCTGAAGGGATCCCAGTGGCTGAGGTGGAATGGTTGAAA |
| SEQ ID NO:3 | GTCTTACTCCAGTGTCGACCACCTGAAGGGATCCCAGTGGCTGAGGTGGAATGGTTGAAA |
| | ************************************************************ |
| | 763 822 |
| AF055634 | AATGAAGACATAATTGATCCCGTTGAAGATCGGAATTTTTATATTACTATTGATCACAAC |
| UNC5C_exon5 | AATGAAGACATAATTGATCCCGTTGAAGATCGGAATTTTTATATTACTATTGATCACAAC |
| SEQ ID NO:1 | AATGAAGACATAATTGATCCCGTTGAAGATCGGAATTTTTATATTACTATTGATCACAAC |
| SEQ ID NO:3 | AATGAAGACATAATTGATCCCGTTGAAGATCGGAATTTTTATATTACTATTGATCACAAC |
| | ************************************************************ |
| | 823 882 |
| AF055634 | CTCATCATAAAGCAGGCCCGACTCTCTGATACTGCAAATTACACCTGTGTTGCCAAAAAC |
| UNC5C_exon5 | CTCATCATAAAGCAGGCCCGACTCTCTGATACTGCAAATTACACCTGTGTTGCCAAAAAC |
| SEQ ID NO:1 | CTCATCATAAAGCAGGCCCGACTCTCTGATACTGCAAATTACACCTGTGTTGCCAAAAAC |
| SEQ ID NO:3 | CTCATCATAAAGCAGGCCCGACTCTCTGATACTGCAAATTACACCTGTGTTGCCAAAAAC |
| | ************************************************************ |
| | 883 942 |
| AF055634 | ATTGTTGCCAAGAGGAAAAGTACAACTGCCACTGTCATAGTCTATGTCAACGGTGGCTGG |
| UNC5C_exon5 | ATTGTTGCCAAGAGGAAAAGTACAACTGCCACTGTCATAGTCTATG |
| UNCSC_exon6 | TCAACGGTGGCTGG |
| SEQ ID NO:1 | ATTGTTGCCAAGAGGAAAAGTACAACTGCCACTGTCATAGTCTATGTCAACGGTGGCTGG |
| SEQ ID NO:3 | ATTGTTGCCAAGAGGAAAAGTACAACTGCCACTGTCATAGTCTATGTCAACGGTGGCTGG |
| | ************************************************************ |
| | 943 1002 |
| AF055634 | TCCACCTGGACGGAGTGGTCTGTGTGTAACAGCCGCTGTGGACGAGGGTATCAGAAACGT |
| UNC5C_exon6 | TCCACCTGGACGGAGTGGTCTGTGTGTAACAGCCGCTGTGGACGAGGGTATCAGAAACGT |
| SEQ ID NO:1 | TCCACCTGGACGGAGTGGTCTGTGTGTAACAGCCGCTGTGGACGAGGGTATCAGAAACGT |
| SEQ ID NO:3 | TCCACCTGGACGGAGTGGTCTGTGTGTAACAGCCGCTGTGGACGAGGGTATCAGAAACGT |
| | ************************************************************ |
| | 1003 1062 |
| AF055634 | ACAAGGACTTGTACCAACCCGGCACCACTCAATGGGGGTGCCTTCTGTGAAGGGCAGAGT |
| UNC5C_exon6 | ACAAGGACTTGTACCAACCCGGCACCACTCAATGGGGGTGCCTTCTGTGAAGGGCAGAGT |
| SEQ ID NO:1 | ACAAGGACTTGTACCAACCCGGCACCACTCAATGGGGGTGCCTTCTGTGAAGGGCAGAGT |
| SEQ ID NO:3 | ACAAGGACTTGTACCAACCCGGCACCACTCAATGGGGGTGCCTTCTGTGAAGGGCAGAGT |
| | ************************************************************ |
| | 1063 1122 |
| AF055634 | GTGCAGAAAATAGCCTGTACTACGTTATGCCCAGTGGATGGCAGGTGGACGCCATGGAGC |
| UNC5C_exon6 | GTGCAGAAAATAGCCTGTACTACGTTATGCCCAG |
| UNC5C_exon7 | TGGATGGCAGGTGGACGCCATGGAGC |
| SEQ ID NO:1 | GTGCAGAAAATAGCCTGTACTACGTTATGCCCAGTGGATGGCAGGTGGACGCCATGGAGC |
| SEQ ID NO:3 | GTGCAGAAAATAGCCTGTACTACGTTATGCCCAGTGGATGGCAGGTGGACGCCATGGAGC |
| | ************************************************************ |
| | 1123 1182 |
| AF055634 | AAGTGGTCTACTTGTGGAACTGAGTGCACCCACTGGCGCAGGAGGGAGTGCACGGCGCCA |
| UNC5C_exon7 | AAGTGGTCTACTTGTGGAACTGAGTGCACCCACTGGCGCAGGAGGGAGTGCACGGCGCCA |
| SEQ ID NO:1 | AAGTGGTCTACTTGTGGAACTGAGTGCACCCACTGGCGCAGGAGGGAGTGCACGGCGCCA |
| SEQ ID NO:3 | AAGTGGTCTACTTGTGGAACTGAGTGCACCCACTGGCGCAGGAGGGAGTGCACGGCGCCA |
| | ************************************************************ |
| | 1183 1242 |
| AF055634 | GCCCCCAAGAATGGAGGCAAGGACTGCGACGGCCTCGTCTTGCAATCCAAGAACTGCACT |
| UNC5C_exon7 | GCCCCCAAGAATGGAGGCAAGGACTGCGACGGCCTCGTCTTGCAATCCAAGAACTGCACT |
| SEQ ID NO:1 | GCCCCCAAGAATGGAGGCAAGGACTGCGACGGCCTCGTCTTGCAATCCAAGAACTGCACT |
| SEQ ID NO:3 | GCCCCCAAGAATGGAGGCAAGGACTGCGACGGCCTCGTCTTGCAATCCAAGAACTGCACT |
| | ************************************************************ |
| | 1243 1302 |
| AF055634 | GATGGGCTTTGCATGCAGA----------------------------------------- |
| UNC5C_exon7 | GATGGGCTTTGCATGCAGA |
| Fusion spec exon | GTTTCATTTATCCCATTTCAACTGAACAGAGAACCCAGAAT |
| SEQ ID NO:1 | GATGGGCTTTGCATGCAGAGTTTCATTTATCCCATTTCAACTGAACAGAGAACCCAGAAT |
| SEQ ID NO:3 | GATGGGCTTTGCATGCAGAGTTTCATTTATCCCATTTCAACTGAACAGAGAACCCAGAAT |
| | ************************************************************ |
| | 1303 1362 |
| AF055634 | ----------------CTGCTCCTGATTCAGATGATGTTGCTCTCTATGTTGGGATTGTG |
| Fusion spec exon | GAATATGGATTTTCTT |
| UNC5C_exon8 | CTGCTCCTGATTCAGATGATGTTGCTCTCTATGTTGGGATTGTG |
| SEQ ID NO:1 | GAATATGGATTTTCTTCTGCTCCTGATTCAGATGATGTTGCTCTCTATGTTGGGATTGTG |
| SEQ ID NO:3 | GAATATGGATTTTCTTCTGCTCCTGATTCAGATGATGTTGCTCTCTATGTTGGGATTGTG |
| | ************************************************************ |
| | 1363 1422 |
| AF055634 | ATAGCAGTGATCGTTTGCCTGGCGATCTCTGTAGTTGTGGCCTTGTTTGTGTATCGGAAG |
| UNC5C_exon8 | ATAGCAGTGATCGTTTGCCTGGCGATCTCTGTAGTTGTGGCCTTGTTTGTGTATCGGAAG |
| SEQ ID NO:1 | ATAGCAGTGATCGTTTGCCTGGCGATCTCTGTAGTTGTGGCCTTGTTTGTGTATCGGAAG |
| SEQ ID NO:3 | ATAGCAGTGATCGTTTGCCTGGCGATCTCTGTAGTTGTGGCCTTGTTTGTGTATCGGAAG |
| AAC90914 | TTGTTTGTGTATCGGAAG |
| | ************************************************************ |
| | 1423 1482 |
| AF055634 | AATCATCGTGACTTTGAGTCAGATATTATTGACTCTTCGGCACTCAATGGGGGCTTTCAN |
| UNC5C_exon8 | AATCATCGTGACTTTGAGTCAGATATTATTGACTCTTCGGCACTCAATGGGGGCTTTCAG |
| SEQ ID NO:1 | AATCATCGTGACTTTGAGTCAGATATTATTGACTCTTCGGCACTCAATGGGGGCTTTCAG |
| SEQ ID NO:3 | AATCATCGTGACTTTGAGTCAGATATTATTGACTCTTCGGCACTCAATGGGGGCTTTCAG |
| AAC90914 | AATCATCGTGACTTTGAGTCAGATATTATTGACTCTTCGGCACTCAATGGGGGCTTTCAG |
| | ************************************************************ |
| | 1483 1542 |
| AF055634 | CCTGTGAACATCAAGGCAGCAAGACAAGATCTGCTGGCTGTACCCCCAGACCTCACGTCA |
| UNC5C_exon8 | CCTGTGAACATCAAGGCAGCAAGACAAG |
| UNC5C_exon9 | ATCTGCTGGCTGTACCCCCAGACCTCACGTCA |
| SEQ ID NO:1 | CCTGTGAACATCAAGGCAGCAAGACAAGATCTGCTGGCTGTACCCCCAGACCTCACGTCA |
| SEQ ID NO:3 | CCTGTGAACATCAAGGCAGCAAGACAAGATCTGCTGGCTGTACCCCCAGACCTCACGTCA |
| AAC90914 | CCTGTGAACATCAAGGCAGCAAGACAAGATCTGCTGGCTGTACCCCCAGACCTCACGTCA |
| | ************************************************************ |
| | 1543 1602 |
| AF055634 | GCTGCAGCCATGTACAGAGGACCTGTCTATGCCCTGCATGACGTCTCAGACAAAATCCCA |
| UNC5C_exon9 | GCTGCAGCCATGTACAGAGGACCTGTCTATGCCCTGCATGACGTCTCAGACAAAATCCCA |
| SEQ ID NO:1 | GCTGCAGCCATGTACAGAGGACCTGTCTATGCCCTGCATGACGTCTCAGACAAAATCCCA |
| SEQ ID NO:3 | GCTGCAGCCATGTACAGAGGACCTGTCTATGCCCTGCATGACGTCTCAGACAAAATCCCA |
| AAC90914 | GCTGCAGCCATGTACAGAGGACCTGTCTATGCCCTGCATGACGTCTCAGACAAAATCCCA |
| | ************************************************************ |
| | 1603 1662 |
| AF055634 | ATGACCAACTCTCCAATTCTGGATCCACTGCCCAACCTGAAAATCAAAGTGTACAACACC |
| UNC5C_exon9 | ATGACCAACTCTCCAATTCTGGATCCACTGCCCAACCTGAAAATCAAAGTGTACAACACC |
| SEQ ID NO:1 | ATGACCAACTCTCCAATTCTGGATCCACTGCCCAACCTGAAAATCAAAGTGTACAACACC |
| SEQ ID NO:3 | ATGACCAACTCTCCAATTCTGGATCCACTGCCCAACCTGAAAATCAAAGTGTACAACACC |
| AAC90914 | ATGACCAACTCTCCAATTCTGGATCCACTGCCCAACCTGAAAATCAAAGTGTACAACACC |
| | ************************************************************ |
| | 1663 1722 |
| AF055634 | TCAGGTGCTGTCTCCCCCCAAGATGACCTCTCTGAGTTTACGTCCAAGCTGTCCCCTCAG |
| UNC5C_exon9 | TCAGGTGCTGTCACCCCCCAAGATGACCTCTCTGAGTTTACGTCCAAGCTGTCCCCTCAG |
| SEQ ID NO:1 | TCAGGTGCTGTCACCCCCCAAGATGACCTCTCTGAGTTTACGTCCAAGCTGTCCCCTCAG |
| SEQ ID NO:3 | TCAGGTGCTGTCACCCCCCAAGATGACCTCTCTGAGTTTACGTCCAAGCTGTCCCCTCAG |
| AAC90914 | TCAAGTGCTGTCACCCCCCAAGATGACCTCTCTGAGTTTACGTCCAAGCTGTCCCCTCAG |
| | *** ******** *********************************************** |
| | 1723 1782 |
| AF055634 | ATGACCCAGTCGTTGTTGGAGAATGAAGCCCTCAGCCTGAAGAACCAGAGTCTAGCAAGG |
| UNC5C_exon9 | ATGACCCAGTCGTTGTTGGAGAATGAAGCCCTCAGCCTGAAGAACCAGAGTCTAGCAAGG |
| SEQ ID NO:1 | ATGACCCAGTCGTTGTTGGAGAATGAAGCCCTCAGCCTGAAGAACCAGAGTCTAGCAAGG |
| SEQ ID NO:3 | ATGACCCAGTCGTTGTTGGAGAATGAAGCCCTCAGCCTGAAGAACCAGAGTCTAGCAAGG |
| AAC90914 | ATGACCCAGTCGTTGTTGGAGAATGAAGCCCTCAGCCTGAAGAACCAGAGTCTAGCAAGG |
| | ************************************************************ |
| | 1783 1842 |
| AF055634 | CAGACTGATCCATCCTGTACCGCATTTGGCAGCTTCAACTCGCTGGGAGGTCACCTTATT |
| UNC5C_exon9 | CAGACTGATCCATCCTGTACCGCATTTGGCAGCTTCAACTCGCTGGGAGGTCACCTTATT |
| SEQ ID NO:1 | CAGACTGATCCATCCTGTACCGCATTTGGCAGCTTCAACTCACTGGGAGGTCACCTTATT |
| SEQ ID NO:3 | CAGACTGATCCATCCTGTACCGCATTTGGCAGCTTCAACTCACTGGGAGGTCACCTTATT |
| AAC90914 | CAGACTGATCCATCCTGTACCGCATTTGGCAGCTTCAACTCGCTGGGAGGTCACCTTATT |
| | ***************************************** ****************** |
| | 1843 1902 |
| AF055634 | GTTCCCAATTCAGGAGTCAGCTTGCTGATTCCCGCTGGGGCCATTCCCCAAGGGAGAGTC |
| UNC5C_exon9 | GTTCCCAATTCAG |
| UNC5C_exon10 | GAGTCAGCTTGCTGATTCCCGCTGGGGCCATTCCCCAAGGGAGAGTC |
| SEQ ID NO:1 | GTTCCCAATTCAGGAGTCAGCTTGCTGATTCCCGCTGGGGCCATTCCCCAAGGGAGAGTC |
| SEQ ID NO:3 | GTTCCCAATTCAGGAGTCAGCTTGCTGATTCCCGCTGGGGCCATTCCCCAAGGGAGAGTC |
| AAC90914 | GTTCCCAATTCAGGAGTCAGCTTGCTGATTCCCGCTGGGGCCATTCCCCAAGGGAGAGTC |
| | ************************************************************ |
| | 1903 1962 |
| AF055634 | TACGAAATGTATGTGACTGTACACAGGAAAGAAACTATGAGGCCACCCATGGATGACTCT |
| UNC5C_exon10 | TACGAAATGTATGTGACTGTACACAGGAAAGAAACTATGAG |
| UNC5C_exon11 | GCCACCCATGGATGACTCT |
| SEQ ID NO:1 | TACGAAATGTATGTGACTGTACACAGGAAAGAAACTATGAGGCCACCCATGGATGACTCT |
| SEQ ID NO:3 | TACGAAATGTATGTGACTGTACACAGGAAAGAAACTATGAGGCCACCCATGGATGACTCT |
| AAC90914 | TACGAAATGTATGTGACTGTACACAGGAAAGAAACTATGAGGCCACCCATGGATGACTCT |
| | ************************************************************ |
| | 1963 2022 |
| AF055634 | CAGACACTTTTGACCCCTGTGGTGAGCTGTGGGCCCCCAGGAGCTCTGCTCACCCGCCCC |
| UNC5C_exon11 | CAGACACTTTTGACCCCTGTGGTGAGCTGTGGGCCCCCAGGAGCTCTGCTCACCCGCCCA |
| SEQ ID NO:1 | CAGACACTTTTGACCCCTGTGGTGAGCTGTGGGCCCCCAGGAGCTCTGCTCACCCGCCCC |
| SEQ ID NO:3 | CAGACACTTTTGACCCCTGTGGTGAGCTGTGGGCCCCCAGGAGCTCTGCTCACCCGCCCC |
| AAC90914 | CAGACACTTTTGACCCCTGTGGTGAGCTGTGGGCCCCCAGGAGCTCTGCTCACCCGCCCC |
| | *********************************************************** |
| | 2023 2082 |
| AF055634 | GTCGTCCTCACTATGCATCACTGCGCAGACCCCAATACCGAGGACTGGAAAATACTGCTC |
| UNC5C_exon11 | GTCGTCCTCACTATGCATCACTGCGCAGACCCCAATACCGAGGACTGGAAAATACTGCTC |
| SEQ ID NO:1 | GTCGTCCTCACTATGCATCACTGCGCAGACCCCAATACCGAGGACTGGAAAATACTGCTC |
| SEQ ID NO:3 | GTCGTCCTCACTATGCATCACTGCGCAGACCCCAATACCGAGGACTGGAAAATACTGCTC |
| AAC90914 | GTCGTCCTCACTATGCATCACTGCGCAGACCCCAATACCGAGGACTGGAAAATACTGCTC |
| | ************************************************************ |
| | 2083 2142 |
| AF055634 | AAGAACCAGGCAGCACAGGGACAGTGGGAGGATGTGGTGGTGGTCGGGGAGGAAAACTTC |
| UNC5C_exon11 | AAGAACCAGGCAGCACAGGGACAGTGGGAG |
| UNC5C_exon12 | GATGTGGTGGTGGTCGGGGAGGAAAACTTC |
| SEQ ID NO:1 | AAGAACCAGGCAGCACAGGGACAGTGGGAGGATGTGGTGGTGGTCGGGGAGGAAAACTTC |
| SEQ ID NO:3 | ------------------------------------------------------------ |
| AAC90914 | ------------------------------------------------------------ |
| | 2143 2202 |
| AF055634 | ACCACCCCCTGCTACATTAAGCTGGATGCAGAGGCCTGCCACATCCTCACAGAGAACCTC |
| UNC5C_exon12 | ACCACCCCCTGCTACATTCAGCTGGATGCAGAGGCCTGCCACATCCTCACAGAGAACCTC |
| SEQ ID NO:1 | ACCACCCCCTGCTACATTCAGCTGGATGCAGAGGCCTGCCACATCCTCACAGAGAACCTC |
| SEQ ID NO:3 | ------------------------------------------------------------ |
| AAC90914 | ------------------------------------------------------------ |
| | 2203 2262 |
| AF055634 | AGCACCTACGCCCTGGTAGGACATTCCACCACCAAAGCGGCTGCAAAGCGCCTCAAGCTG |
| UNC5C_exon12 | AGCACCTACGCCCTGGTAGGACATTCCACCACCAAAGCGGCTGCAAAGCGCCTCAAGCTG |
| SEQ ID NO:1 | AGCACCTACGCCCTGGTAGGACATTCCACCACCAAAGCGGCTGCAAAGCGCCTCAAGCTG |
| SEQ ID NO:3 | ------------------------------------------------------------ |
| AAC90914 | ------------------------------------------------------------ |
| | 2263 2322 |
| AF055634 | GCCATCTTTGGGCCCCTGTGCTGCTCCTCGCTGGAGTACAGCATCCGAGTCTACTGTCTG |
| UNC5C_exon12 | GCCATCTTTGGGCCCCTGTGCTGCTCCTCGCTGGAGTACAGCATCCGAGTCTACTGTCTG |
| SEQ ID NO:1 | GCCATCTTTGGGCCCCTGTGCTGCTCCTCGCTGGAGTACAGCATCCGAGTCTACTGTCTG |
| SEQ ID NO:3 | --------------------------CTCGCTGGAGTACAGCATCCGAGTCTACTGTCTG |
| AAC90914 | --------------------------CTCGCTGGAGTACAGCATCCGAGTCTACTGTCTG |
| | ********************************** |
| | 2323 2382 |
| AF055634 | GATGACACCCAGGATGCCCTGAAGGAAATTTTACATCTTGAGAGACAGACGGGAGGACAG |
| UNC5C_exon12 | GATGACACCCAGGATGCCCTGAAG |
| UNC5C_exon13 | GAAATTTTACATCTTGAGAGACAGACGGGAGGACAG |
| SEQ ID NO:1 | GATGACACCCAGGATGCCCTGAAGGAAATTTTACATCTTGAGAGACAGACGGGAGGACAG |
| SEQ ID NO:3 | GATGACACCCAGGATGCCCTGAAGGAAATTTTACATCTTGAGAGACAGACGGGAGGACAG |
| AAC90914 | GATGACACCCAGGATGCCCTGAAGGAAATTTTACATCTTGAGAGACAGACGGGAGGACAG |
| | ************************************************************ |
| | 2383 2442 |
| AF055634 | CTCCTAGAAGAACCTAAGGCTCTTCATTTTAAAGGCAGCACCCACAACCTGCGCCTGTCA |
| UNC5C_exon13 | CTCCTAGAAGAACCTAAGGCTCTTCATTTTAAAGGCAGCACCCACAACCTGCGCCTGTCA |
| SEQ ID NO:1 | CTCCTAGAAGAACCTAAGGCTCTTCATTTTAAAGGCAGCACCCACAACCTGCGCCTGTCA |
| SEQ ID NO:3 | CTCCTAGAAGAACCTAAGGCTCTTCATTTTAAAGGCAGCACCCACAACCTGCGCCTGTCA |
| AAC90914 | CTCCTAGAAGAACCTAAGGCTCTTCATTTTAAAGGCAGCACCCACAACCTGCGCCTGTCA |
| | ************************************************************ |
| | 2443 2502 |
| AF055634 | ATTCACGATATCGCCCATTCCCTCTGGAAGAGCAAATTGCTGGCTAAATATCAGGAAATT |
| UNC5C_exon13 | ATTCACGATATCGCCCATTCCCTCTGGAAGAGCAAATTGCTGGCTAAATATCAG |
| UNC5C_exon14 | GAAATT |
| SEQ ID NO:1 | ATTCACGATATCGCCCATTCCCTCTGGAAGAGCAAATTGCTGGCTAAATATCAGGAAATT |
| SEQ ID NO:3 | ATTCACGATATCGCCCATTCCCTCTGGAAGAGCAAATTGCTGGCTAAATATCAGGAAATT |
| AAC90914 | ATTCACGATATCGCCCATTCCCTCTGGAAGAGCAAATTGCTGGCTAAATATCAGGAAATT |
| | ************************************************************ |
| | 2503 2562 |
| AF055634 | CCATTTTACCATGTTTGGAGTGGATCTCAAAGAAACCTGCACTGCACCTTCACTCTGGAA |
| UNC5C_exon14 | CCATTTTACCATGTTTGGAGTGGATCTCAAAGAAACCTGCACTGCACCTTCACTCTGGAA |
| SEQ ID NO:1 | CCATTTTACCATGTTTGGAGTGGATCTCAAAGAAACCTGCACTGCACCTTCACTCTGGAA |
| SEQ ID NO:3 | CCATTTTACCATGTTTGGAGTGGATCTCAAAGAAACCTGCACTGCACCTTCACTCTGGAA |
| AAC90914 | CCATTTTACCATGTTTGGAGTGGATCTCAAAGAAACCTGCACTGCACCTTCACTCTGGAA |
| | ************************************************************ |
| | 2563 2622 |
| AF055634 | AGATTTAGCCTGAACACAGTGGAGCTGGTTTGCAAACTCTGTGTGCGGCAGGTGGAAGGA |
| UNC5C_exon14 | AGATTTAGCCTGAACACAGTGGAGCTGGTTTGCAAACTCTGTGTGCGGCAGGTGGAAGGA |
| SEQ ID NO:1 | AGATTTAGCCTGAACACAGTGGAGCTGGTTTGCAAACTCTGTGTGCGGCAGGTGGAAGGA |
| SEQ ID NO:3 | AGATTTAGCCTGAACACAGTGGAGCTGGTTTGCAAACTCTGTGTGCGGCAGGTGGAAGGA |
| AAC90914 | AGATTTAGCCTGAACACAGTGGAGCTGGTTTGCAAACTCTGTGTGCGGCAGGTGGAAGGA |
| | ************************************************************ |
| | 2623 2682 |
| AF055634 | GAAGGGCAGATCTTCCAGCTCAACTGCACCGTGTCAGAGGAACCTACTGGCATCGATTTG |
| UNC5C_exon14 | GAAGGGCAGATCTTCCAGCTCAACTGCACCGTGTCAGAG |
| UNC5C_eXon15 | GAACCTACTGGCATCGATTTG |
| SEQ ID NO:1 | GAAGGGCAGATCTTCCAGCTCAACTGCACCGTGTCAGAGGAACCTACTGGCATCGATTTG |
| SEQ ID NO:3 | GAAGGGCAGATCTTCCAGCTCAACTGCACCGTGTCAGAGGAACCTACTGGCATCGATTTG |
| AAC90914 | GAAGGGCAGATCTTCCAGCTCAACTGCACCGTGTCAGAGGAACCTACTGGCATCGATTTG |
| | ************************************************************ |
| | 2683 2742 |
| AF055634 | CCGCTGCTGGATCCTGCGAACACCATCACCACGGTCACGGGGCCCAGTGCTTTCAGCATC |
| UNC5C_exon15 | CCGCTGCTGGATCCTGCGAACACCATCACCACGGTCACGGGGCCCAGTGCTTTCAGCATC |
| SEQ ID NO:1 | CCGCTGCTGGATCCTGCGAACACCATCACCACGGTCACGGGGCCCAGTGCTTTCAGCATC |
| SEQ ID NO:3 | CCGCTGCTGGATCCTGCGAACACCATCACCACGGTCACGGGGCCCAGTGCTTTCAGCATC |
| AAC90914 | CCGCTGCTGGATCCTGCGAACACCATCACCACGGTCACGGGGCCCAGTGCTTTCAGCATC |
| | ************************************************************ |
| | 2743 2802 |
| AF055634 | CCTCTCCCTATCCGGCAGAAGCTCTGTAGCAGCCTGGATGCCCCCCAGACGAGAGGCCAT |
| UNC5C_exon15 | CCTCTCCCTATCCGGCAGAAGCTCTGTAGCAGCCTGGATGCCCCCCAGACGAGAGGCCAT |
| SEQ ID NO:1 | CCTCTCCCTATCCGGCAGAAGCTCTGTAGCAGCCTGGATGCCCCCCAGACGAGAGGCCAT |
| SEQ ID NO:3 | CCTCTCCCTATCCGGCAGAAGCTCTGTAGCAGCCTGGATGCCCCCCAGACGAGAGGCCAT |
| AAC90914 | CCTCTCCCTATCCGGCAGAAGCTCTGTAGCAGCCTGGATGCCCCCCAGACGAGAGGCCAT |
| | ************************************************************ |
| | 2803 2862 |
| AF055634 | GACTGGAGGATGCTGGCCCATAAGCTGAACCTGGACAGGTACTTGAATTACTTTGCCACC |
| UNC5C_exon15 | GACTGGAGGATGCTGGCCCATAAGCTGAACCTGGACAG |
| UNC5C_exon16 | GTACTTGAATTACTTTGCCACC |
| SEQ ID NO:1 | GACTGGAGGATGCTGGCCCATAAGCTGAACCTGGACAGGTACTTGAATTACTTTGCCACC |
| SEQ ID NO:3 | GACTGGAGGATGCTGGCCCATAAGCTGAACCTGGACAGGTACTTGAATTACTTTGCCACC |
| AAC90914 | GACTGGAGGATGCTGGCCCATAAGCTGAACCTGGACAGGTACTTGAATTACTTTGCCACC |
| | ************************************************************ |
| | 2863 2922 |
| AF055634 | AAATCCAGCCCAACTGGCGTAATCCTGGATCTTTGGGAAGCACAGAACTTCCCAGATGGA |
| UNC5C_exon16 | AAATCCAGCCCAACTGGCGTAATCCTGGATCTTTGGGAAGCACAGAACTTCCCAGATGGA |
| SEQ ID NO:1 | AAATCCAGCCCAACTGGCGTAATCCTGGATCTTTGGGAAGCACAGAACTTCCCAGATGGA |
| SEQ ID NO:3 | AAATCCAGCCCAACTGGCGTAATCCTGGATCTTTGGGAAGCACAGAACTTCCCAGATGGA |
| AAC90914 | AAATCCAGCCCAACTGGCGTAATCCTGGATCTTTGGGAAGCACAGAACTTCCCAGATGGA |
| | ************************************************************ |
| | 2923 2982 |
| AF055634 | AACCTGAGCATGCTGGCAGCTGTCTTGGAAGAAATGGGAAGACATGAAACGGTGGTGTCC |
| UNC5C_exon16 | AACCTGAGCATGCTGGCAGCTGTCTTGGAAGAAATGGGAAGACATGAAACGGTGGTGTCC |
| SEQ ID NO:1 | AACCTGAGCATGCTGGCAGCTGTCTTGGAAGAAATGGGAAGACATGAAACGGTGGTGTCC |
| SEQ ID NO:3 | AACCTGAGCATGCTGGCAGCTGTCTTGGAAGAAATGGGAAGACATGAAACGGTGGTGTCC |
| AAC90914 | AACCTGAGCATGCTGGCAGCTGTCTTGGAAGAAATGGGAAGACATGAAACGGTGGTGTCC |
| | ************************************************************ |
| | 2983 3003 3028 3042 |
| AF055634 | TTAGCAGCAGAAGGGCAGTATTAACCACCATGCTGGAAGGGGAAATGCAGTATTAACCAC |
| UNC5C_exon16 | TTAGCAGCAGAAGGGCAGTATTAACCACCATGCTGGAAGGGGAAATGCAGTATTAACCAC |
| SEQ ID NO:1 | TTAGCAGCAGAAGGGCAGTATTAACCACCATGCTGGAAGGGGAAAT-------------- |
| SEQ ID NO:3 | TTAGCAGCAGAAGGGCAGTATTAACCACCATGCTGGAAGGGGAAAT-------------- |
| AAC90914 | TTAGCAGCAGAAGGGCAGTAT--------------------------------------- |
| | ************************************************************ |
| | 3043 3102 |
| AF055634 | CATGCTGGAAGGGGAAATGAAGGACAAAAATGCACAGGGAGTCTGTGGCCGTCCAGGTGA |
| UNC5C_exon16 | CATGCTGGAAGGGGAAATGAAGGACAAAAATGCACAGGGAGTCTGTGGCCGTCCAGGTGA |
| | ************************************************************ |
| | 3103 3162 |
| AF055634 | ATCACAGCTGAGGAGGAAATCCAGATGAGACCAATGCACTTCACAGGCAAGAATGCAGCA |
| UNC5C_exon16 | ATCACAGCTGAGGAGGAAATCCAGATGAGACCAATGCACTTCACAGGCAAGACTGCAGCA |
| | **************************************************** ******* |
| | 3163 3222 |
| AF055634 | GGAGCCAGAAGGAAAACAGATACAACTGCCCATGTACATGCCCACTTTACTCGGAGATCA |
| UNC5C_exon16 | GGAGCCAGAAGGAAAACAGATACAACTGCCCATGTACATGCCCACTTTACTCGGACATCA |
| | ******************************************************* **** |
| | 3223 3282 |
| AF055634 | TCACGGGAGTTAAGAAAAATTGTGTAAATTTGTACCTTGAATTTAGCTATCAACCTAATT |
| UNC5C_exon16 | TCACGGGAGTTAAGAAAAATTGTGTAAATTTGTACCTTGAATTTAGCTATCAACCTAATT |
| | ************************************************************ |
| | 3283 3342 |
| AF055634 | TTCCTCTTAGTTGGGCTGTATGCTGTGTGGTACAGGATCTTACAGTTTCCTAGGAAACGC |
| UNC5C_exon16 | TTCCTCTTAGTTGGGCTGTATGCTGTGTGGTACAGGATCTTACAGTTTCCTAGGAAACGC |
| | ************************************************************ |
| | 3343 3402 |
| AF055634 | TTTTTATTGCTATCCAGATATATGGATAAACTTTCTTAACAAACCCAATTTCTACAAATG |
| UNC5C_exon16 | TTTTTATTGCTATCCAGATATATGGATAAACTTTCTTAACAAACCCAATTTCTACAAATG |
| | ************************************************************ |
| | 3403 3462 |
| AF055634 | TTGTTTACATCAAATTGGACAGGGATGCAGACACTGTCCATGGCTCGTTCTATTTTTGTT |
| UNC5C_exon16 | TTGTTTACATCAAATTGGACAGGGATGCAGACACTGTCCATGGCTCGTTCTATTTTTGTT |
| | ************************************************************ |
| | 3463 3522 |
| AF055634 | CAAATCATTTGAAGTTGAAGCTGTGGACGGTTTGTTGTGTCTATTTCAGATTAGTAATTT |
| UNC5C_exon16 | CAAATCATTTGAAGTTGAAGCTGTGGACGGTTTGTTGTGTCTATTTCAGATTAGTAATTT |
| | ************************************************************ |
| | 3523 3582 |
| AF055634 | ACAGAGAAATCACAGACTTTTGCTACAAATCGTGTGCATCAAGTGTCTCAGATAATCCTC |
| UNC5C_exon16 | ACAGAGAAATCACAGACTTTTGCTACAAATCGTGTGCATCAAGTGTCTCAGATAATCCTC |
| | ************************************************************ |
| | 3583 3642 |
| AF055634 | CCATCAGTGTTCTGTTTCTAGAACTTGTAGAACCAGTGTTACTGTTTGTATCAGGGAAGT |
| UNC5C_exon16 | CCATCAGTGTTCTGTTTCTAGAACTTGTAGAACCAGTGTTACTGTTTGTATCAGGGAAGT |
| | ************************************************************ |
| | 3643 3702 |
| AF055634 | GGAGAATCTAAGTGTAAAAAAGAAATAACTAAGACTCCTATTCCTTGGAGGGACCCTTCT |
| UNC5C_exon16 | GGAGAATCTAAGTGTAAAAAAGAAATAACTAAGACTCCTATTCCTTGGAGGGACCCTTCT |
| | ************************************************************ |
| | 3703 3732 |
| AF055634 | GGTGCCCTTTGGGAATAAAGCTGTAGCACTGC |
| UNC5C_exon16 | GGTGCCCTTTGGGAATAAAGCTGTAGCACTGC |
| | ******************************** |

Table 2 below shows a protein alignment of the 1RORα1-UNC5C (SEQ ID NO: 2) and sRORα1-UNC5C (SEQ ID NO: 4) fusion proteins with UNC5C:

TABLE 2

| | |
|---|---|
| | 1 59 |
| UNC5C exon 1 | --------------MRKGLRATAARCGLGLGYLLQMLVLPALALLSASGTGSAAQ |
| UNC5C | --------------MRKGLRATAARCGLGLGYLLQMLVLPALALLSASGTGSAAQDDDF |
| SEQ ID NO:2 | MESAPAAPDPAASEPGSSGADAAAGSRETPLNQESARKSEPPAPVRRQSYSSTSRDDDF |
| SEQ ID NO:4 | MESAPAAPDPAASEPGSSGADAAAGSRETPLNQESARKSEPPAPVRRQSYSSTSRDDDF |
| RORa1 exon 1 | MESAPAAPDPAASEPGSSGADAAAGSRETPLNQESARKSEPPAPVRRQSYSSTSR |
| | **** |
| | 60 119 |
| UNC5C | FHELPETFPSDPPEPLPHFLIEPEEAYIVKNKPVNLYCKASPATQIYFKCNSEWVHQKDH |
| SEQ ID NO:2 | FHELPETFPSDPPEPLPHFLIEPEEAYIVKNKPVNLYCKASPATQIYFKCNSEWVHQKDH |
| SEQ ID NO:4 | FHELPETFPSDPPEPLPHFLIEPEEAYIVKNKPVNLYCKASPATQIYFKCNSEWVHQKDH |
| | ************************************************************ |
| | 120 179 |
| UNC5C | IVDERVDETSGLIVREVSIEISRQQVEELFGPEDYWCQCVAWSSAGTTKSRKAYVRIAYL |
| SEQ ID NO:2 | IVDERVDETSGLIVREVSIEISRQQVEELFGPEDYWCQCVAWSSAGTTKSRKAYVRIAYL |
| SEQ ID NO:4 | IVDERVDETSGLIVREVSIEISRQQVEELFGPEDYWCQCVAWSSAGTTKSRKAYVRIAYL |
| | ************************************************************ |
| | 180 239 |
| UNC5C | RKTFEQEPLGKEVSLEQEVLLQCRPPEGIPVAEVEWLKNEDIIDPVEDRNFYITIDHNLI |
| SEQ ID NO:2 | RKTFEQEPLGKEVSLEQEVLLQCRPPEGIPVAEVEWLKNEDIIDPVEDRNFYITIDHNLI |
| SEQ ID NO:4 | RKTFEQEPLGKEVSLEQEVLLQCRPPEGIPVAEVEWLKNEDIIDPVEDRNFYITIDHNLI |
| | ************************************************************ |
| | 240 299 |
| UNC5C | IKQARLSDTANYTCVAKNIVAKRKSTTATVIVYVNGGWSTWTEWSVCNSRCGRGYQKRTR |
| SEQ ID NO:2 | IKQARLSDTANYTCVAKNIVAKRKSTTATVIVYVNGGWSTWTEWSVCNSRCGRGYQKRTR |
| SEQ ID NO:4 | IKQARLSDTANYTCVAKNIVAKRKSTTATVIVYVNGGWSTWTEWSVCNSRCGRGYQKRTR |
| | ************************************************************ |
| | 300 359 |
| UNC5C | TCTNPAPLNGGAFCEGQSVQKIACTTLCPVDGRWTPWSKWSTCGTECTHWRRRECTAPAP |
| SEQ ID NO:2 | TCTNPAPLNGGAFCEGQSVQKIACTTLCPVDGRWTPWSKWSTCGTECTHWRRRECTAPAP |
| SEQ ID NO:4 | TCTNPAPLNGGAFCEGQSVQKIACTTLCPVDGRWTPWSKWSTCGTECTHWRRRECTAPAP |
| | ************************************************************ |
| | 360 419 |
| UNC5C | KNGGKDCDGLVLQSKNCTDGLCMQ-------------------TAPDSDDVALYVGIVIA |
| SEQ ID NO:2 | KNGGKDCDGLVLQSKNCTDGLCMQSFIYPISTEQRTQNEYGFSSAPDSDDVALYVGIVIA |
| SEQ ID NO:4 | KNGGKDCDGLVLQSKNCTDGLCMQSFIYPISTEQRTQNEYGFSSAPDSDDVALYVGIVIA |
| Fusion specific | SFIYPISTEQRTQNEYGYS |
| exon | ************************ ***************** |
| | 420 479 |
| UNC5C | VIVCLAISVVVALFVYRKNHRDFESDIIDSSALNGGFQPVNIKAARQDLLAVPPDLTSAA |
| SEQ ID NO:2 | VIVCLAISVVVALFVYRKNHRDFESDIIDSSALNGGFQPVNIKAARQDLLAVPPDLTSAA |
| SEQ ID NO:4 | VIVCLAISVVVALFVYRKNHRDFESDIIDSSALNGGFQPVNIKAARQDLLAVPPDLTSAA |
| | ************************************************************ |
| | 480 539 |
| UNC5C | AMYRGPVYALHDVSDKIPMTNSPILDPLPNLKIKVYNTSGAVSPQDDLSEFTSKLSPQMT |
| SEQ ID NO:2 | AMYRGPVYALHDVSDKIPMTNSPILDPLPNLKIKVYNTSGAVTPQDDLSEFTSKLSPQMT |
| SEQ ID NO:4 | AMYRGPVYALHDVSDKIPMTNSPILDPLPNLKIKVYNTSGAVTPQDDLSEFTSKLSPQMT |
| | ****************************************** ***************** |
| | 540 599 |
| UNC5C | QSLLENEALSLKNQSLARQTDPSCTAFGSFNSLGGHLIVPNSGVSLLIPAGAIPQGRVYE |
| SEQ ID NO:2 | QSLLENEALSLKNQSLARQTDPSCTAFGSFNSLGGHLIVPNSGVSLLIPAGAIPQGRVYE |
| SEQ ID NO:4 | QSLLENEALSLKNQSLARQTDPSCTAFGSFNSLGGHLIVPNSGVSLLIPAGAIPQGRVYE |
| | ************************************************************ |
| | 600 659 |
| UNC5C | MYVTVHRKETMRPPMDDSQTLLTPVVSCGPPGALLTRPVVLTMHHCADPNTEDWKILLKN |
| SEQ ID NO:2 | MYVTVHRKETMRPPMDDSQTLLTPVVSCGPPGALLTRPVVLTMHHCADPNTEDWKILLKN |
| SEQ ID NO:4 | MYVTVHRKETMRPPMDDSQTLLTPVVSCGPPGALLTRPVVLTMHHCADPNTEDWKILLLA |
| | ********************************************************** |
| | 660 719 |
| UNC5C | QAAQGQWEDVVVVGEENFTTPCYIKLDAEACHILTENLSTYALVGHSTTGAAAKRLKLAI |
| SEQ ID NO:2 | QAAQGQWEDVVVVGEENFTTPCYIQLDAEACHILTENLSTYALVGHSTTGAAAKRLKLAI |
| SEQ ID NO:4 | GVQHPSLLSG |
| | 720 779 |
| UNC5C | FGPLCCSSLEYSIRVYCLDDTQDALKEILHLERQTGGQLLEEPKALHFKGSTHNLRLSIH |
| SEQ ID NO:2 | FGPLCCSSLEYSIRVYCLDDTQDALKEILHLERQTGGQLLEEPKALHFKGSTHNLRLSIH |
| | 780 839 |
| UNC5C | DIAHSLWKSKLLAKYQEIPFYHVWSGSQRNLHCTFTLERFSLNTVELVCKLCVRQVEGEG |
| SEQ ID NO:2 | DIAHSLWKSKLLAKYQEIPFYHVWSGSQRNLHCTFTLERFSLNTVELVCKLCVRQVEGEG |
| | 840 899 |
| UNC5C | QIFQLNCTVSEEPTGIDLPLLDPANTITTVTGPSAFSIPLPIRQKLCSSLDAPQTRGHDW |
| SEQ ID NO:2 | QIFQLNCTVSEEPTGIDLPLLDPANTITTVTGPSAFSIPLPIRQKLCSSLDAPQTRGHDW |
| | 900 959 |
| UNC5C | RMLAHKLNLKRYLNYFATKSSPTGVILDLWEAQNFPDGNLSMLAAVLEEMGRHETVVSLA |
| SEQ ID NO:2 | RMLAHKLNLKRYLNYFATKSSPTGVILDLWEAQNFPDGNLSMLAAVLEEMGRHETVVSLA |
| | 960 |
| UNC5C | AEGQY |
| SEQ ID NO:2 | AEGQY |

As used herein “RORα1-UNC5C” refers to both the short and large fusion protein, unless otherwise stated.

The genomic DNA sequence at the fusion site between chromosome 4 (nucleotide positions 1-3221) and chromosome 15 (nucleotide positions 3229-6277) isolated from the translocation 4:15 is set out as SEQ ID NO:11. Nucleotide positions 3222-3228 of SEQ ID NO:11 are a 7 bp insertion created in the translocation event. The localisation of this sequence is shown in FIG. 4.

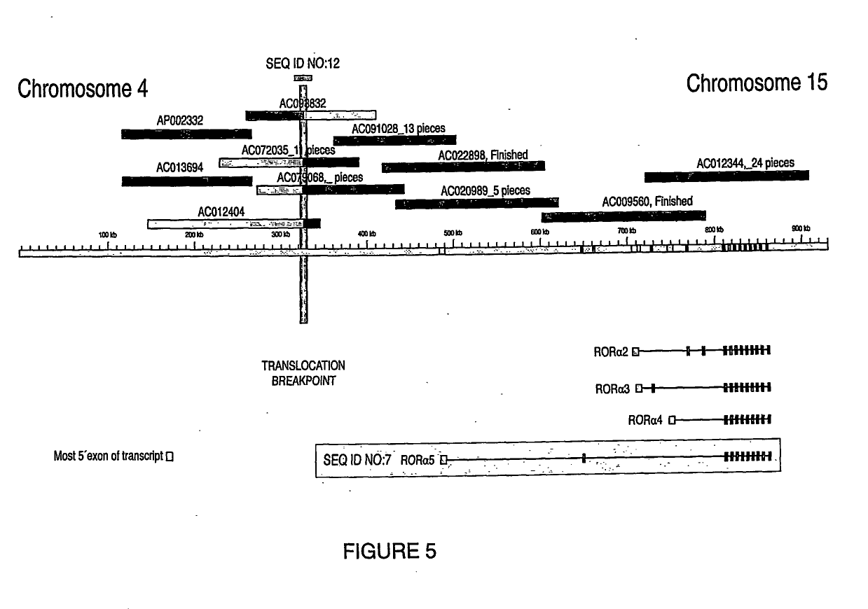

The genomic DNA sequence at the fusion site between chromosome 15 (nucleotide positions 1-998) and chromosome 4 (nucleotide positions 1036-1829) isolated from the translocation 15:4 is set out as SEQ ID NO:12. Nucleotide positions 999-1035 of SEQ ID NO: 12 are a 36 bp insertion created in the translocation event. The localisation of this sequence is shown in FIG. 5.

A cDNA encoding a novel isoform of RORα, designated “RORα5”, is set out as SEQ ID NO: 7 (with the first nucleotide of the RORα5 coding region accorded position 188), and its gene product is set out at SEQ ID NO: 8.

According to one aspect of the present invention there is provided a polypeptide selected from the group consisting of:
i) a fusion peptide of RORα1-UNC5C polypeptide comprising SEQ ID NO:2 or a fragment thereof of at least 10 amino acids wherein the fragment comprises either the junction of the fusion protein, the flanking regions of the fusion specific exon or the fusion specific exon of SEQ ID NO:2;
ii) a fusion polypeptide of RORα1-UNC5C polypeptide comprising SEQ ID NO:4 or a fragment thereof of at least 10 amino acids wherein the fragment comprises either the junction of the fusion protein, the flanking regions of the fusion specific exon or the fusion specific exon of SEQ ID:4.
iii) a polypeptide of RORα5 polypeptide comprising the amino acid sequence of SEQ ID NO:8 or a fragment thereof of at least 10 amino acids
   or an amino acid sequence at least 90%, preferably 95%, 98% or 99% identical to either i) or ii) or iii) or a complement thereof.

In a preferred embodiment, a conservative analogue of the protein is contemplated.

In this specification conservative amino acid analogues of specific amino acid sequences are contemplated which retain the relevant biological properties of a component of the invention but differ in sequence by one or more conservative amino acid substitutions, deletions or additions. However the specifically listed amino acid sequences are preferred. Typical conservative amino acid substitutions are tabulated below.

CONSERVATIVE SUBSTITUTIONS

| | | Preferred |
|---|---|---|
| Original | Exemplary Substitutions | Substitutions |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; | Ile |
| | Ala; Phe | |
| Lys (K) | Arg; Gln; Asn | Arg |
| Met (M) | Leu; Phe; Ile | Leu |
| Phe (F) | Leu; Val; Ile; Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp; Phe; Thr; Ser | Phe |
| Val (V) | Ile; Leu; Met; Phe; Ala; | Leu |
| | Norleucine | |

In this specification nucleic acid variations (deletions, substitutions and additions) of specific nucleic acid sequences are contemplated which retain which the ability to hybridise under stringent conditions to the specific sequence in question. Stringent conditions are defined as 6×SSC, 0.1% SDS at 60° C. for 5 minutes. However specifically listed nucleic acid sequences are preferred. It is contemplated that peptide nucleic acid may be an acceptable equivalent of polynucleotide sequences, at least for purposes that do not require translation into protein (Wittung (1994) Nature 368, 561).

By “the junction of the fusion protein”, we mean that the fragment of at least 10 amino acids includes the amino acid accorded position 55 and 56 of SEQ ID NO:2 or SEQ ID NO:4.

By “the flanking regions of the fusion specific exon” we mean that the fragment of at least 10 amino acids includes either the amino acids accorded position 383 and 384 or the amino acids accorded position 403 and 404 of SEQ ID NO:2 or SEQ ID NO:4

The invention further features an isolated nucleic acid molecule capable of encoding the RORα1-UNC5C polypeptide or a fragment thereof or the RORα5 polypeptide or a fragment thereof as defined above or a complement thereof, or a nucleic acid molecule having at least 65% preferably 75%, 85%, 90%, 95%, 98% or 99%, identity thereto. Preferred nucleotide sequences are SEQ ID NO:1, 3 or 7.

The invention further features a vector comprising the nucleic acid molecule as defined above.

Also included within the invention is the vector as defined above, wherein said vector is an expression vector.

The invention further features a host cell comprising the expression vector as defined above.

The invention further features a method of making the RORα1-UNC5C polypeptide or the RORα5 polypeptide as defined above comprising: incubating the host cell as defined hereinabove in a medium conducive to expression of the polypeptide and optionally purifying the polypeptide.

The invention further features an antibody specific for the RORα1-UNC5C polypeptide or the RORα5 polypeptide as defined above.

The invention further features a method for detecting the presence of the RORα1-UNC5C polypeptide or the RORα5 polypeptide as defined above in a biological sample comprising:
contacting the sample with a binding agent capable of specifically binding to the polypeptide, and measuring the amount of binding.

The invention further features a method of detecting the presence of the isolated nucleic acid molecule as defined above comprising:
contacting said nucleic acid with a nucleic acid capable of specifically hybridizing to the translocation breakpoint.

Regions of specificity in the fusion protein/gene include, for example, the junction point and fusion specific insert.

In yet another aspect, the invention features an isolated nucleic acid molecule comprising a nucleotide sequence having at least 65% identity to a degenerate variant of SEQ ID NO: 1, SEQ ID NO: 3 or SEQ ID NO: 7. The invention further features an isolated nucleic acid molecule comprising the complement of SEQ ID NO: 1, SEQ ID NO: 3 or SEQ ID NO: 7.

The invention further features an isolated nucleic acid encoding a RORα1-UNC5C polypeptide, the isolated nucleic acid molecule comprising a nucleotide sequence having at least 65% identity to a degenerate variant of SEQ ID NO: 1 or SEQ ID NO: 3.

The invention further features an isolated nucleic acid encoding a RORα5 polypeptide, the isolated nucleic acid molecule comprising a nucleotide sequence having at least 65% identity to a degenerate variant of SEQ ID NO: 7.

The invention further features a vector comprising the nucleic acid molecule of any of the above and a host cell comprising the vector.

Also included within the invention is an isolated nucleic acid molecule encoding a protein having 65% sequence identity to the amino acid sequence of SED ID NO:2, SEQ ID NO: 4 or SEQ ID NO: 9. Moreover the invention features a purified polypeptide comprising an amino acid sequence having at least 65% identity to the amino acid sequence of SEQ ID NO: 2, SEQ ID NO: 4 or SEQ ID NO: 9.

Also within the invention is a method for producing a protein comprising culturing the host cell having SEQ ID NO:1, SEQ ID NO: 3 or SEQ ID NO:7 under conditions whereby said protein is produced, and recovering the protein from the host cell culture.

The invention further includes a method for detecting a polynucleotide which encodes a protein comprising the amino acid sequence of SEQ ID NO:2, SEQ ID NO: 4 or SEQ ID NO: 8 in a biological sample comprising the steps of:

a) hybridizing the complement of the polynucleotide sequence which encodes SEQ ID NO: 2, SEQ ID NO: 4 or SEQ ID NO: 8 to a nucleic acid material of a biological sample, thereby forming a hybridization complex; and

b) detecting the hybridization complex, wherein the presence of the complex correlates with the presence of a polynucleotide encoding a protein in the biological sample.

One aspect of the invention is directed to a method for detecting the presence of other obesity susceptibility genes which may be responsible for the development of obesity by determining the identity of genes in and around the resulting translocation junction t(4:15) or t(15:4). The method includes determining the identity of genes in and around the translocation junction, e.g., located 20 cM, or less, adjacent to the junction and determining if the gene shows aberrant expression compared to the gene in a person not having the chromosomal translocation.

As used herein, an “obesity susceptibility gene” or “obesity gene” is a gene which as a consequence of the translocation event between chromosome 4 and chromosome 15 has a predisposing influence in the development of obesity in a subject. For example, an obesity susceptibility gene can refer to any or all of the following genes: UNC5C, sUNC5C, RORα1, RORα5, 1RORα1-UNC5C and sRORα1-UNC5C.

As used herein “obesity protein” or “obesity susceptibility polypeptide” is the gene product of the obesity susceptibility gene or obesity gene.

In yet another aspect, the invention features a method for detecting the chromosomal translocation comprising analysing a sample of DNA from an individual for the presence of the translocation breakpoint, wherein the presence of the translocation breakpoint indicates that the person has a susceptibility to developing obesity. The nucleic acid sequence of the translocation junctions t(4:15) and t(15:4) is set out in SEQ ID NO: 11 and SEQ ID NO: 12 respectively. The localisation of these sequences are shown in FIGS. 4 and 5 respectively.

The invention further includes a method for identifying a test compound that modulates the expression of an obesity susceptibility gene such as UNC5C, sUNC5C, RORα1, RORα1, 1RORα1-UNC5C and sRORα1-UNC5C identified in the methods above, comprising contacting a cell capable of expressing an obesity susceptibility gene with a test compound; and determining the level of expression of the obesity susceptibility gene in the presence of the test compound, wherein a decrease or an increase in the obesity susceptibility gene expression, as compared to the level of expression of the obesity susceptibility gene in the absence of the compound, is indicative that the test compound modulates the expression of the obesity susceptibility gene.

Also within the invention is a method of identifying a test compound that modulates the activity of a protein encoded by an obesity susceptibility gene such as UNC5C, sUNC5C, RORα1, RORα5, 1RORα1-UNC5C and sRORα1-UNC5C, comprising contacting the protein with a test compound and determining the level of activity of the obesity protein in the presence of the compound, wherein a decrease or an increase in protein activity is indicative that the test compound modulates protein activity.

The invention further features a method of treating a subject having obesity comprising administering an effective amount of the compound identified above. The invention further includes a pharmaceutical composition comprising the compound identified as above, and a pharmaceutically acceptable adjuvant, diluent or carrier.

The invention further includes a method of making a pharmaceutical composition. In one aspect, the method includes: contacting a cell capable of expressing an obesity susceptibility gene with a test compound; determining the level of expression of the obesity susceptibility gene identified above in the presence of the test compound, wherein a decrease or an increase in expression of the obesity susceptibility gene, as compared to the level of expression of the obesity susceptibility gene in the absence of the compound, is indicative that the test compound modulates the obesity susceptibility gene expression; and formulating the test compound that modulates the obesity susceptibility gene expression into a pharmaceutical composition.

In another aspect, the invention includes making a pharmaceutical composition including contacting an obesity susceptibility protein with a test compound; determining the level of activity of the obesity susceptibility protein in the presence of the compound, wherein a decrease in the presence of the obesity susceptibility protein, as compared to the level of activity of the obesity susceptibility protein in the absence of the compound, is indicative that the test compound modulates obesity susceptibility protein activity; and formulating the test compound that decreases obesity susceptibility protein activity into a pharmaceutical composition.

In another aspect, the invention includes a method for determining if an obesity susceptibility gene identified above has an altered level of gene expression comprising comparing the level of obesity gene expression in a cell from a patient having obesity with a control cell, and determining the level of expression of the obesity susceptibility gene in both cells, wherein a decrease or an increase in expression of the obesity susceptibility gene, as compared to the level of expression of the obesity susceptibility gene in the control cell, indicates that the obesity susceptibility gene has altered gene expression.

The invention further includes methods of diagnosing obesity or a susceptibility thereto in a subject. The method includes determining the level of an obesity susceptibility gene mRNA and/or the level of an obesity causing protein in a sample from a subject; and comparing the level of the mRNA and/or protein in the sample with a control, wherein a decrease or an increase in the level of the mRNA and/or protein in the sample compared to the control indicates that the subject has obesity, or a susceptibility thereto. The invention also extends to products useful for carrying out the assay, such as DNA probes (labelled or unlabelled), kits and the like.

The invention further includes methods of diagnosing obesity or a susceptibility thereto in a subject. The method includes analysing for the presence of RORα1-UNC5C mRNA and/or protein in a sample from a subject; wherein the presence of the RORα1-UNC5C mRNA and/or protein indicates that the subject has obesity, or a susceptibility thereto. The invention also extends to products useful for carrying out the assay, such as DNA probes (labelled or unlabelled), kits and the like.

The present invention also includes gene-based therapies directed at obesity. Therapies may be in the form of polynucleotides comprising all or a portion of the obesity susceptibility gene(s), or obesity gene locus, placed in appropriate vectors or delivered to target cells in direct ways which would modify the function of the obesity protein.

General molecular biology techniques are described in “Current Protocols in Molecular is Biology Volumes 1-3, edited by F M Asubel, R Brent and R E Kingston; published by John Wiley, 1998 and Sambrook, J. and Russell, D. W., Molecular Cloning: A Laboratory Manual, the third edition, Cold Spring Harbor Laboratory Press, Cold Spring Harbor, N.Y., 2001.

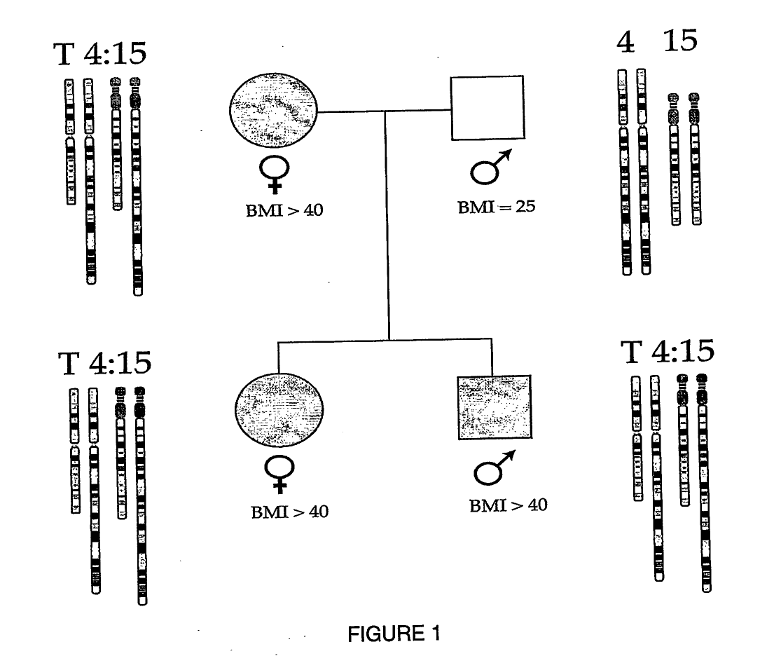

FIG. 1 shows a pedigree of the family with schematically drawn chromosomes showing the inheritance of the balanced translocation where all affected individuals are extremely obese. BMI=body mass index.

FIG. 2 shows the normal gene map of human chromosome 4 at the translocation breakpoint.

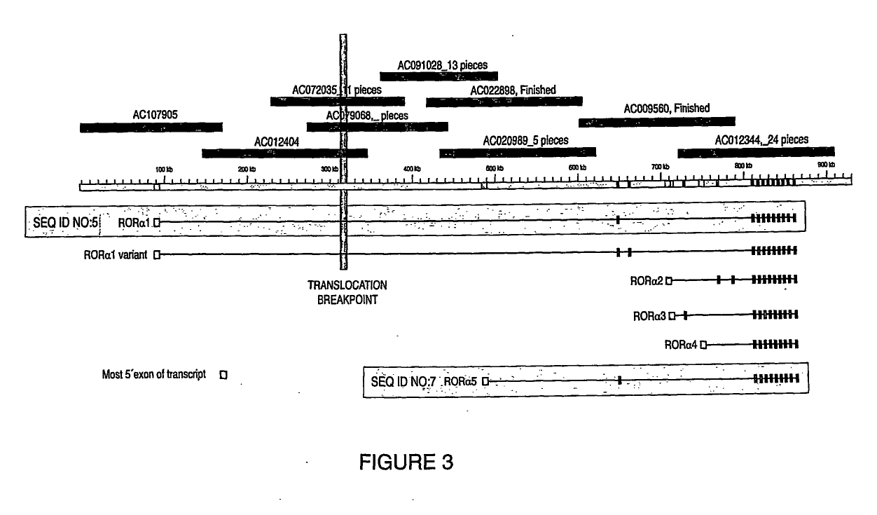

FIG. 3 shows the normal map of chromosome 15 at the translocation breakpoint with the RORα locus.

FIG. 4 shows the outcome of the translocation event at chromosome 4 (translocation chromosome 4:15) with the resulting fusion transcripts.

FIG. 5 shows the outcome of the translocation event at chromosome 15 (translocation chromosome 15:4) with the destruction of RORα1.

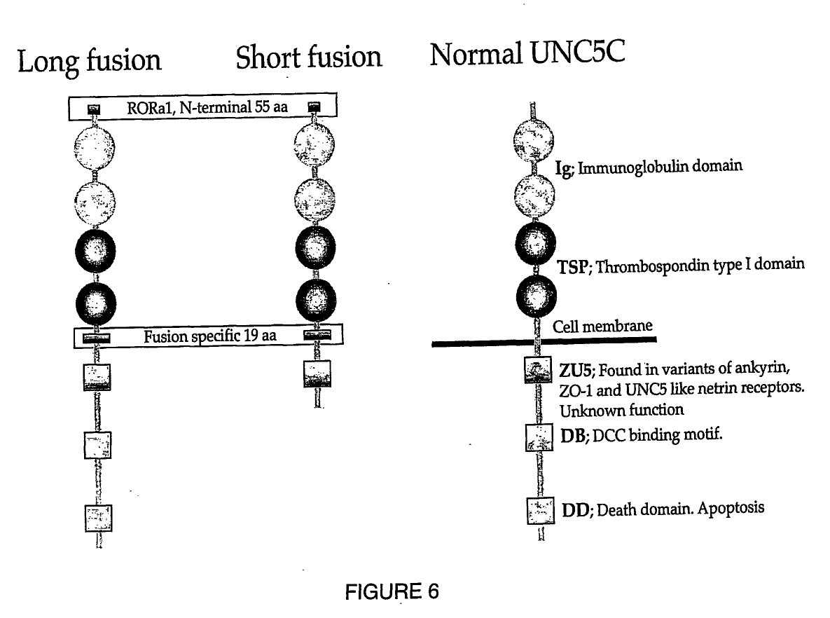

FIG. 6 shows a schematic view of the protein domain architecture of the 1RORα1-UNC5C and sRORα1-UNC5C fusion protein transcripts together with that of normal human UNC5C.

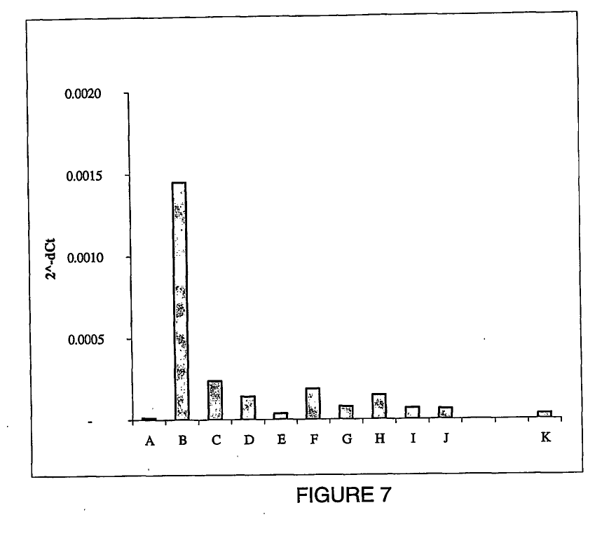

FIG. 7 shows Taqman™ analysis of RORα5 expression in various tissues, with the highest expression being found in brain. FIG. 7 shows RORα5 expression levels where the X axis labelling indicates the following tissues; A=bone marrow, B=brain, C=heart, D=kidney, E=liver, F=lung, G=testis, H=skeletal muscle, I=small intestine, J=spleen and K=fat.

FIG. 8 shows a schematic view of the 1RORα1-UNC5C and sRORα1-UNC5C fusion transcripts together with UNC5C and sUNC5C.

The invention is based on the finding that a genetic alteration in patients with obesity is the translocation of two specific chromosomes (t(4;15)). This event leads to the production of two fusion proteins that is thought to promote the development of obesity. It also destroys one copy of RORα1 (SEQ ID NOS: 5 & 6) thus maybe reducing the expression of this gene in certain tissues during development. It may also affect the expression of the other isoforms of RORa, in particular RORα5 (SEQ ID NOS: 7 & 8) which is the closest situated isoform of RORα on the other side of the breakpoint at chromosome 15.

The breakpoint in chromosome 4 occurs at cytoband 4q22.3 and the breakpoint in chromosome 15 occurs at cytoband 15q22.2. The balanced translocation results in patients having one normal chromosome 4 and one normal chromosome 15 together with the reciprocal translocation chromosomes. The outcome of the translocation event is that the expression and\or activity of at least one or more genes is affected.

The breakpoint at chromosome 4 is between the genes Pyruvate dehydrogense A2 (PDHA2) and UNC5C (SEQ ID NOS: 9 & 10). While neither of these genes are destroyed in the mutation event, UNC5C was found to be upregulated in adipocytes from obese affected individuals, as compared with control adipocytes.

UNC5 is required for correct projections of pioneer axons and for migrating cells along the body wall in C. elegans (Leung Hagensteijn et. al., Cell 1992 October 71 (2): 289-99). UNC5 genetically interacts with UNC6 and UNC40. Mutants of all three genes defects in the axon projections. UNC5 and UNC40 are transmembrane receptors expressed on developing neurons. Both are receptors for the diffusible protein UNC6/Netrin (Hedgecock et. al.; Neuron 1990 January; 4(1): 61-85) (Wadsworth; Trends Neurosci 2002 August; 25(8): 423-9) Vertebrates have at least 3 UNC5 like genes named UNC5A, UNC5B and UNC5C (Leonardo et. al.; Nature 1997 April; 386 (6627): 833-8) and the protein contains two immunoglobulin domains, two trombospondin domains on the extracellular part and intracellular domains ZU5, DB and a Death Domain (Leung Hagensteijn et. al., Cell 1992 October 71 (2): 289-99) (Keleman & Dickson; Neuron 2001 November; 32(4): 605-17). Human UNC5C is localised on chromosome 4 at cytoband q22.3 (Ackerman & Knowles; Genomics 1998 September; 52(2): 205-8).

The breakpoint at chromosome 15 is within the first intron of RORα1. This isoform of RORα is destroyed by the mutation event. Moreover, it was found that the translocation resulted in a novel transcript, which is a fusion between RORα1 exon 1 with UNC5C exon 2 producing 2 novel fusion proteins through differential splicing within the UNC5C gene (SEQ ID NO:2 and SEQ ID NO: 4).

RORα1 is an orphan nuclear hormone receptor localized on chromosome 15 q22.3 (V. Giguere et. al. Genomics 1995 August; 28(3): 596-8). Nuclear hormone receptors (NHR) constitute a super family of highly conserved intracellular receptors found in nematodes, flies and vertebrates. The protein architecture is a N-terminal transactivation domain followed by a DNA binding domain, hinge region and a c-terminal ligand binding domain. Many NHRs form dimers upon activation and translocate into the nucleus where they bind DNA to activate or repress target gene expression. There are four publicly available isoforms of RORα1, namely RORα1-4.

We have identified yet another isoform of RORα, designated RORα5 (SEQ ID 7 & 8). RORα5 was isolated with 5′RACE (rapid amplification of C-terminal ends) performed on public available adipocyte cDNA library (clontech). The expression pattern of RORα5 is similar to RORα1, and is most abundantly expressed in the brain (FIG. 7).

Accordingly, the present invention describes the chromosomal location of obesity susceptibility genes and provides methods for detecting the presence of such genes. The present invention enables the development of novel therapies for obesity by screening for compounds and other entities, such as antibodies, which modulate the activity of the proteins encoded by the associated genes, e.g., UNC5C, sUNC5C, RORα1, RORα5, 1RORα1-UNC5C and sRORα1-UNC5C. Knowledge of the sequence of the obesity susceptibility genes also enables the development of novel antigene methods to modulate the expression of the obesity susceptibility gene and also enables the development of novel gene therapy techniques to treat obesity. The discovery of obesity susceptibility genes may also assist in developing novel methods for diagnosing obesity via (i) measuring the levels of the translated mRNA present in affected tissue and (ii) measuring the levels of the protein in affected tissue. It is possible that the diagnosis of obesity, or the susceptibility of an individual to obesity, by these methods may be achieved in patients who do not yet display the classical symptoms of the disease. Such determination of susceptibility to obesity or the early detection of disease development may lead to earlier clinical intervention, than is currently possible, and may lead to more effective treatment of the disease.

Further Identification of Obesity Susceptibility Genes

The present findings describes a chromosomal translocation which alters the expression\activity of genes in and around the translocation junction causing obesity. The genetic location of such genes are at the breakpoint of a translocation between chromosome 4q22.3 and chromosome 15q22.2. Thus, the present invention includes methods of identifying genes around the translocation breakpoint and determining if any gene around the translocation breakpoint has abherrant expression. The invention includes identifying genes, e.g., 8, 7, 6, 5, 4, 3, 2 or 1 cM around the translocation breakpoint and comparing the expression profile of these genes against the control genes. This can be done by using techniques known in the art to further analyze and delineate this genetic region. For example, using adipocytes isolated from obese individuals carrying the translocation and control adipocytes it is possible to compare the expression profile of an identified obese susceptibility gene identified in the region, or the activity of the gene product of the gene and determine abherrant expression or activity.

Techniques such as Northern analysis, in situ hybridization or expression profiling can be used to further verify the association of these genes with obesity. In one example, a reporter-based assay may be devised to detect whether an identified obesity susceptibility gene has a different transcription levels and/or message stability compared to the same gene in a person not susceptibile to obesity. Individuals who carry the obesity susceptibility gene may exhibit differences in their ability to regulate protein biosynthesis under different physiological conditions and may display altered physiological abilities.

In another example, the level of obesity gene expression can be assayed by detecting and measuring obesity transcription. For example, RNA from adipocytes from a person carrying the translocation can be isolated and tested utilizing hybridization or PCR techniques. Such analyses can reveal both quantitative and qualitative aspects of the expression pattern of the obesity gene, including activation or inactivation of obesity gene expression.

Pathway Mapping

Also within the invention is the identification and elucidation of the obesity biochemical\signal transduction pathway of which the obesity susceptibility protein is a component. In particular, the invention includes identifying the other components of the obesity susceptibility protein biochemical pathway. In this way it is possible to identify the specific critical signaling pathway which links the disease stimulus to the cell's response and enables the identification of new potential targets for therapy intervention.

As used herein, a obesity susceptibility protein “target molecule” is a molecule in the obesity biochemical pathway with which the obesity susceptibility protein binds or interacts, directly or indirectly, with, or is a molecule that regulates the expression of the obesity gene or translation of the protein in nature. For example, the target molecule can be a protein which directly interacts with the obesity susceptibility protein, or can be a protein which does not itself directly interact with the obesity susceptibility protein, but which is a component of the obesity susceptibility protein biochemical pathway. In one example, the target molecule can be an intercellular protein or a protein which facilitates the association of downstream or upstream signaling molecules with the obesity susceptibility protein.

According to a further aspect of the invention there is provided the use of the obesity susceptibility protein in research to identify further gene targets implicated in obesity. Methods for identifying proteins which interact with the obesity susceptibility protein are known in the art, e.g., the two-hybrid assay (see, e.g., U.S. Pat. No. 5,283,317; Zervos et al. Cell 72:223-232, 1993) or using cell culture techniques to identify binding partners.

The two-hybrid system is, based on the modular nature of most transcription factors, which consist of separable DNA-binding and activation domains. Briefly, the assay utilizes two different DNA constructs. In one construct, the gene that codes for an obesity susceptibility protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an obesity susceptibility protein-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the “target” protein which interacts with the obesity susceptibility protein.

Alternatively, binding partners for the obesity susceptibility protein can be identified using cell culture techniques or using cells obtained directly from an obesity patient. The method includes isolating the obesity susceptibility protein of interest from the cell and determining the identity of its target molecule. Initial screening can be accomplished by Western blot analysis to analyse immunochemically, using antibodies against the obesity susceptibility protein, the size of the obesity susceptibility protein-target molecule complex. Further analysis of the complex will reveal the identity of the target molecule.

The gene and protein encoded by the target molecule is also a potential target for therapeutic intervention in obesity disease, for instance in the development of antisense nucleic acid targeted to the mRNA; or more widely in the identification or development of chemical or hormonal therapeutic agents. The person skilled in the art is also capable of devising screening assays to identify compounds (chemical or biological) that modulate (activate or inhibit) the identified gene, which compounds may prove useful as therapeutic agents in treating or preventing obesity.

RORα1-UNC5C and RORα5 Nucleic Acid Sequence and Polypeptide

The invention encompasses a RORα1-UNC5C nucleic acid sequence having at least 65%, 70%, 75%, 80%, 85%, 90% or 99%, sequence identity to the RORα1-UNC5C nucleic acid sequence of SEQ ID NO: 1 or SEQ ID NO: 3. The invention also encompasses a RORα5 nucleic acid sequence having at least 65%, 70%, 75%, 80%, 85%, 90% or 99%, sequence identity to the RORα5 nucleic acid sequence of SEQ ID NO: 7. The comparison of sequences and determination of percent sequence identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at ttp://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The invention also encompasses polynucleotides which encode the RORα1-UNC5C protein of SEQ ID NO: 2 or SEQ ID NO: 4, or the RORα5 protein of SEQ ID NO: 8. It will be appreciated by those skilled in the art that as a result of the degeneracy of the genetic code, a multitude of nucleotide sequences encoding RORα1-UNC5C polypeptide or RORα5 polypeptide, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequence of naturally occurring RORα1-UNC5C polypeptide or RORα5 polypeptide, and all such variations are to be considered as being specifically disclosed.

The invention also encompasses production of DNA sequences, or fragments thereof, which encode 1RORα1-UNC5C, sRORα1-UNC5C or RORα5 polypeptide and their derivatives, entirely by synthetic chemistry. After production, the synthetic sequence may be inserted into any of the many available expression vectors and cell systems using reagents that are well known in the art.

Also encompassed by the invention are polynucleotide sequences that are capable of hybridizing to the claimed RORα1-UNC5C or RORα5 nucleic acid seqeunce, and in particular, those shown in SEQ ID NO: 1, SEQ ID NO: 3 or SEQ ID NO: 7, under various conditions of stringency as taught in Wahl, G. M. and S. L. Berger (1987; Methods Enzymol. 152:399-407) and Kimmel, A. R. (1987; Methods Enzymol. 152:507-511). Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0 SSC at 50° C. to a high stringency of about 0.2×SSC at 50° C. In addition, the temperature in the wash step can be increased from low stringency conditions at room temperature, about 22° C., to high stringency conditions at about 65° C. Moderately stringent conditions are, for example at about 2.0×SSC and about 40° C.

Also included in the invention are RORα1-UNC5C polypeptides having at least 65% amino acid sequence identity to the RORα1-UNC5C polypeptide of SEQ ID NO: 2 or SEQ ID NO: 4 and which retains at least one biological or other functional characteristic or activity of the RORα1-UNC5C polypeptide. A most preferred RORα1-UNC5C variant is one having at least 65% 70%, 80% or 90% amino acid sequence identity to SEQ ID NO: 2 or SEQ ID NO: 4.

Also included in the invention is RORα5 polypeptide having at least 65% amino acid sequence identity to the RORα5 polypeptide of SEQ ID NO: 8 and which retains at least one biological or other functional characteristic or activity of the RORα5 polypeptide. A most preferred RORα5 variant is one having at least 65% 70%, 80% or 90% amino acid sequence identity to SEQ ID NO: 8.

The invention also includes variants of the RORα1-UNC5C polypeptide or the RORα5 polypeptide which can contain one or more substitutions of amino acid residues which result in a silent change and a functionally equivalent RORα1-UNC5C polypeptide or RORα5 polypeptide respectively. Deliberate amino acid substitutions may be made on the basis of similarity in polarity, charge, solubility, hydrophobicity, hydrophilicity, and/or the amphipathic nature of the residues as long as the biological or immunological activity of RORα1-UNC5C polypeptide or RORα5 polypeptide is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine, glycine and alanine, asparagine and glutamine, serine and threonine, and phenylalanine and tyrosine.

Obesity Proteins and Polypeptides

Obesity proteins such as the RORα1, RORα5, UNC5C, sUNC5C or the RORα1-UNC5C fusion proteins can be prepared for a variety of uses, including but not limited to the generation of antibodies, as reagents in diagnostic assays, in the identification of other cellular gene products involved in the pathogenesis of obesity, as reagents in assays for screening for compounds that can be used in the treatment of obesity, and as pharmaceutical reagents useful in the treatment of obesity.

A variety of host-expression vector systems may be utilized to express the obesity nucleotide sequences of the invention. The expression systems that may be used for purposes of the invention include but are not limited to microorganisms such as bacteria (e.g., E. coli, B. subtilis) transformed with recombinant bacteriophage DNA, plasmid DNA or cosmid DNA expression vectors containing obesity nucleotide sequences or mammalian cell systems (e.g., COS, CHO, BHK, 293, 3T3) harbouring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the obesity gene product being expressed. For example, when a large quantity of such a protein is to be produced, for the generation of pharmaceutical compositions of obesity protein or for raising antibodies to the obesity protein, for example, vectors which direct the expression of high levels of fusion protein products that are readily purified may be desirable. Such vectors include, but are not limited to, the E. coli expression vector pUR278 (Ruther et al., EMBO J. 2:1791, 1983), in which the obesity coding sequence may be ligated individually into the vector in frame with the lacZ coding region so that a fusion protein is produced; pIN vectors (Inouye & Inouye, Nucleic Acids Res. 13:3101-3109, 1985; Van Heeke & Schuster, J. Biol. Chem. 264:5503-5509, 1989); and the like. PGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The PGEX vectors are designed to include thrombin or factor Xa protease cleavage sites so that the cloned target gene product can be released from the GST moiety.

In mammalian host cells, a number of viral-based expression systems may be utilized. In cases where an adenovirus is used as an expression vector, the obesity nucleotide sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the obesity gene product in infected hosts. (E.g., See Logan & Shenk, Proc. Natl. Acad. Sci. USA 81:3655-3659, 1984).

For long-term, high-yield production of recombinant proteins, stable expression is preferred. For example, cell lines which stably express the obesity sequences described above may be engineered. Rather than using expression vectors which contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the obesity gene product. Such engineered cell lines may be particularly useful in screening and evaluation of compounds that affect the endogenous activity of the obesity gene product.

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht, et al., Proc. Natl. Acad. Sci. USA 88:8972-8976, 1991). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni.2+. nitriloacetic acid-agarose columns and histidine-tagged proteins are selectively eluted with imidazole-containing buffers.

The obesity gene products can also be expressed in transgenic animals. Animals of any species, including, but not limited to, mice, rats, rabbits, guinea pigs, pigs, micro-pigs, goats, and non-human primates, for example, baboons, monkeys, and chimpanzees may be used to generate obesity transgenic animals.

Any technique known in the art may be used to introduce the obesity transgene into animals to produce the founder lines of transgenic animals. Such techniques include, but are not limited to pronuclear microinjection (U.S. Pat. No. 4,873,191); retrovirus mediated gene transfer into germ lines (Van der Putten et al., Proc. Natl. Acad. Sci. USA 82:6148-6152, 1985); gene targeting in embryonic stem cells (Thompson et al., Cell 56:313-321, 1989); electroporation of embryos (Lo, Mol. Cell. Biol. 3:1803-1814, 1983); and sperm-mediated gene transfer (Lavitrano et al., Cell 57:717-723, 1989); etc. For a review of such techniques, see Gordon, Transgenic Animals, Intl. Rev. Cytol. 115:171-229, 1989, which is incorporated by reference herein in its entirety.

The present invention provides for transgenic animals that carry the obesity transgene in all their cells, as well as animals which carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko, M. et al., Proc. Natl. Acad. Sci. USA 89:6232-6236, 1992). Once transgenic animals have been generated, the expression of the recombinant obesity gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques which include but are not limited to Northern blot analysis of tissue samples obtained from the animal, in situ hybridization analysis, and RT-PCR. Samples of obesity gene-expressing tissue, may also be evaluated immunocytochemically using antibodies specific for the obesity transgene product.

Antibodies to Obesity Proteins

Antibodies that specifically recognize one or more epitopes of obesity proteins, or peptide fragments, are also encompassed by the invention. Such antibodies include but are not limited to polyclonal antibodies, monoclonal antibodies (mAbs), humanized or chimeric antibodies, single chain antibodies, Fab fragments, F(ab′).2 fragments, fragments produced by a Fab expression library, anti-idiotypic (anti-Id) antibodies, and epitope-binding fragments of any of the above.

The antibodies of the invention may be used, for example, in the detection of the obesity in a biological sample and may, therefore, be utilized as part of a diagnostic or prognostic technique whereby patients may be tested for abnormal amounts of obesity. Such antibodies may also be utilized in conjunction with, for example, compound screening schemes for the evaluation of the effect of test compounds on expression and/or activity of the obesity gene product. Additionally, such antibodies can be used in conjunction with the gene therapy techniques described, for example, evaluate the normal and/or engineered obesity-expressing cells prior to their introduction into the patient. Such antibodies may additionally be used as a method for the inhibition of abnormal obesity activity. Thus, such antibodies may, therefore, be utilized as part of obesity treatment methods.

Methods of making and detecting labelled antibodies are well known (Campbell; Monoclonal Antibody Technology, in: Laboratory Techniques in Biochemistry and Molecular Biology, Volume 13. Eds: Burdon R et al. Elsevier, Amsterdam (1984)). The term antibody includes both monoclonal antibodies, which are a substantially homogeneous population, and polyclonal antibodies which are heterogeneous populations. The term also includes inter alia, humanised and chimeric antibodies. Monoclonal antibodies to specific antigens may be obtained by methods known to those skilled in the art, such as from hybridoma cells, phage display libraries or other methods. Monoclonal antibodies may be inter alia, human, rat or mouse derived. For the production of human monoclonal antibodies, hybridoma cells may be prepared by fusing spleen cells from an immunised animal, e.g. a mouse, with a tumour cell. Appropriately secreting hybridoma cells may thereafter be selected (Koehler & Milstein, Nature 256:495-497 (1975); Cole et al., “Monoclonal antibodies and Cancer Therapy”, Alan R Liss Inc, New York N.Y. pp 77-96 (1985)). Such antibodies may be of any immunoglobulin class including IgG, IgM, IgE, IgA, IgD and any subclass thereof.

Polyclonal antibodies can be generated by immunisation of an animal (such as a mouse, rat, goat, horse, sheep etc) with an antigen, such as one of the obesity proteins used in this invention.

Rodent antibodies may be humanised using recombinant DNA technology according to techniques known in the art. Alternatively, chimeric antibodies, single chain antibodies, Fab fragments may also be developed against the polypeptides of the invention (Huse et al., Science 256:1275-1281 (1989)), using skills known in the art. Antibodies so produced have a number of uses which will be evident to the molecular biologist or immunologist skilled in the art. Such uses include, but are not limited to, monitoring enzyme expression, development of assays to measure enzyme activity and use as a therapeutic agent. Enzyme linked immunosorbant assays (ELISAs) are well known in the art and would be particularly suitable for detecting the obesity protein or polypeptide fragments thereof in a test sample.

Screening Assay

The invention also provides a method for identifying modulators, i.e., test compounds (e.g., peptides, peptidomimetics, small molecules or other drugs) which modulate the activity of an obesity susceptibility gene.

In one example, the invention provides methods for screening for test compounds for use in the treatment of obesity by screening for test compounds that modulate the activity of the obesity protein, or a portion thereof. The test compounds of the present invention can be obtained using any of the numerous approaches in combinatorial library methods known in the art, including: biological libraries. The method can be a cell-based method or a cell free method. The screening methods according to the invention may be operated using conventional procedures, for example by bringing the test compound or compounds to be screened and an appropriate substrate into contact with the obesity polypeptide, or a cell capable of producing it, or a cell membrane preparation thereof, and determining affinity for the obesity polypeptide in accordance with standard techniques.

Any compound identified in this way may prove useful in the treatment of obesity in humans and/or other animals. The invention thus extends to a compound selected through its ability to regulate the activity of the obesity protein in vivo as primarily determined in a screening assay utilising an obesity polypeptide or a homologue or fragment thereof, or a gene coding therefore for use in the treatment of a disease in which the over- or under-activity or unregulated activity of the protein is implicated. According to a further aspect of the invention there is provided a screening assay or method for identifying potential anti-obesity therapeutic compounds comprising contacting an assay system capable of detecting the effect of a test compound against expression level of obesity, with a test compound and assessing the change in expression level of obesity. Compounds that modulate the expression of DNA or RNA of obesity polypeptides may be detected by a variety of assay systems. A suitable assay system may be a simple “yes/no” assay to determine whether there is a change in expression of a reporter gene, such as beta-galactosidase, luciferase, green fluorescent protein or others known to the person skilled in the art (reviewed by Naylor, Biochem. Pharmacol. 58:749-57, 1999). The assay system may be made quantitative by comparing the expression or function of a test sample with the levels of expression or function in a standard sample. Systems in which transcription factors are used to stimulate a positive output, such as transcription of a reporter gene, are generally referred to as “one-hybrid systems” (Wang, M. M. and Reed, R. R. Nature 364:121-126, 1993). Using a transcription factor to stimulate a negative output (growth inhibition) may thus be referred to as a “reverse one-hybrid system” (Vidal et al, supra). Therefore, in an embodiment of the present invention, a reporter gene is placed under the control of the obesity promoter.

In a further aspect of the invention we provide a cell or cell line comprising a reporter gene under the control of the obesity promoter.

According to another aspect of the present invention there is provided a method of screening for a compound potentially useful for treatment of obesity which comprises assaying the compound for its ability to modulate the activity or amount of obesity. Preferably the assay is selected from:
i) measurement of obesity activity using a cell line which expresses the obesity polypeptide or using purified obesity polypeptide; and
ii) measurement of obesity transcription or translation in a cell line expressing the obesity polypeptide.

Thus, in a further aspect of the invention, cell cultures expressing the obesity polypeptide can be used in a screen for therapeutic agents. Effects of test compounds may be assayed by changes in mRNA or protein of obesity. As described above, cells (i.e. mammalian, bacterial etc) can be engineered to express the obesity polypeptide.

Thus, according to a further aspect of the invention there is provided a method of testing potential therapeutic agents for the ability to suppress the obesity phenotype comprising contacting a test compound with a cell engineered to express the obesity polypeptide; and determining whether said test compound suppressed expression of the obesity polypeptide.

We also provide a method for identifying inhibitors of transcription of obesity, which method comprises contacting a potential therapeutic agent with a cell or cell line as described above and determining inhibition of obesity transcription by the potential therapeutic agent by reference to a lack of or reduction in expression of the reporter gene.

Any convenient test compound or library of test compounds may be used in conjunction with the test assay. Particular test compounds include low molecular weight chemical compounds (preferably with a molecular weight less than 1500 daltons) suitable as pharmaceutical or veterinary agents for human or animal use, or compounds for non-administered use such as cleaning/sterilising agents or for agricultural use. Test compounds may also be biological in nature, such as antibodies.

According to a further aspect of the invention there is provided a compound identified by a screening method as defined herein.

According to another aspect of the present invention there is provided use of a compound able to modulate the activity or amount of obesity in the preparation of a medicament for the treatment of obesity. Modulation of the amount of obesity by a compound may be brought about for example through altered gene expression level or message stability. Modulation of the activity of obesity by a compound may also be brought about for example through compound binding to the obesity protein. In one embodiment, modulation of obesity comprises use of a compound able to reduce the activity or amount of obesity. In another embodiment, modulation of obesity comprises use of a compound able to increase the activity or amount of obesity.

Diagnostic Test

To determine if an individual is susceptible to obesity determination of the presence of the translocation junction between chromosome 4q22.3 and chromosome 15q22.2 can be performed to predict whether the individual has a high risk obesity chromosome. Moreover, determination of the translocation breakpoint can be used to identify the underlying cause of obesity in a person who is obese.

A diagnostic test method lends itself readily to the formulation of kits which can be utilized in diagnosis. The diagnostic kits may comprise appropriate packaging and instructions for use in the methods of the invention. Such kits may further comprise appropriate buffer(s) and polymerase(s) such as thermostable polymerases, for example taq polymerase.

Methods for the diagnosis of obesity may, for example, utilize reagents such as the nucleotide sequences which span the breakpoint of sRORα1-UNC5C or 1RORα1-UNC5C antibodies. Specifically, such reagents may be used, for example, for: (1) the detection of the presence of short or long RORα1-UNC5C transcript, (2) the detection of perturbations or abnormalities in the signal transduction pathway mediated by RORα1-UNC5C or UNC5C.

For the detection of mutations, any nucleated cell can be used as a starting source for genomic nucleic acid. For the detection of an obesity susceptibility gene such as sRORα1-UNC5C or 1RORα1-UNC5C gene products, any cell type or tissue in which the obesity gene is expressed can be used.

Detection of the Obesity Gene and Transcripts

Obesity susceptibility gene transcripts can be detected by utilizing a number of techniques. Nucleic acid from any nucleated cell can be used as the starting point for such assay techniques, and may be isolated according to standard nucleic acid preparation procedures which are well known to those of skill in the art.

DNA may be used in hybridization or amplification assays of biological samples to detect abnormalities involving obesity gene structure or gene regulatory elements, including point mutations, insertions, deletions and chromosomal rearrangements. Such assays may include, but are not limited to, Southern analyses, single stranded conformational polymorphism analyses (SSCP), and PCR analyses.

Such diagnostic methods for the detection of obesity gene-specific mutations can involve for example, contacting and incubating nucleic acids including recombinant DNA molecules, cloned genes or degenerate variants thereof, obtained from a sample, for example, derived from a patient sample or other appropriate cellular source, with one or more labelled nucleic acid reagents including recombinant DNA molecules, under conditions favourable for the specific annealing of these reagents to their complementary sequences within the obesity gene. Preferably, the lengths of these nucleic acid reagents are at least 15 to 30 nucleotides. After incubation, all non-annealed nucleic acids are removed from the nucleic acid: obesity molecule hybrid. The presence of nucleic acids which have hybridized, if any such molecules exist, is then detected. Using such a detection scheme, the nucleic acid from the cell type or tissue of interest can be immobilized, for example, to a solid support such as a membrane, or a plastic surface such as that on a microtiter plate or polystyrene beads. Detection of the remaining, annealed, labelled obesity nucleic acid reagents is accomplished using standard techniques well-known to those in the art. The obesity gene sequences to which the nucleic acid reagents have annealed can be compared to the annealing pattern expected from a normal obesity gene sequence in order to determine whether an obesity gene mutation is present.

Detection of the Obesity Gene Products

Antibodies directed against wild type or mutant obesity gene products or conserved variants or peptide fragments thereof, can also be used as obesity diagnostics. Such diagnostic methods, can be used to detect abnormalities in the level of obesity gene expression, or abnormalities in the structure and/or temporal, tissue, cellular, or subcellular location of the obesity, and may be performed in vivo or in vitro, such as, for example, on biopsy tissue.

For example, antibodies directed to epitopes of the obesity can be used in vivo to detect the pattern and level of expression of the obesity in the body. Such antibodies can be labelled, and injected into a subject in order to visualize binding to the obesity expressed in the body using methods such as X-rays, CAT-scans, or MRI.

Additionally, any obesity fusion protein or obesity conjugated protein whose presence can be detected, can be administered. For example, obesity fusion or conjugated proteins labelled with a radio-opaque or other appropriate compound can be administered and visualized in vivo, as discussed, above for labelled antibodies.

Alternatively, immunoassays or fusion protein detection assays, as described above, can be utilized on biopsy and autopsy samples in vitro to permit assessment of the expression pattern of the obesity. Such assays are not confined to the use of antibodies that define the obesity, but can include the use of antibodies directed to epitopes of any region of the obesity.

The tissue or cell type to be analyzed will generally include those which are known, or suspected, to express the obesity gene. The protein isolation methods employed herein can, for example, be such as those described in Harlow and Lane (Harlow, E. and Lane, D., 1988, Antibodies: A Laboratory Manual, Cold Spring Harbor Laboratory Press, Cold Spring Harbor, N.Y.), which is incorporated herein by reference in its entirety. The isolated cells can be derived from cell culture or from a patient. The analysis of cells taken from culture may be a necessary step in the assessment of cells that could be used as part of a cell-based gene therapy technique or, alternatively, to test the effect of compounds on the expression of the obesity gene.

Gene Therapy

The invention encompasses gene therapy methods and compositions for treating and preventing obesity.

In one example, the loss of normal obesity gene product function can result in the development of obesity. Increasing obesity gene product activity, or activation of the obesity pathway (e.g., downstream activation) would therefore facilitate progress in individuals exhibiting a deficient level of obesity gene expression and/or obesity activity.

Alternatively, obesity may be ameliorated by decreasing the level of obesity gene expression, and/or obesity gene activity, and/or downregulating activity of the obesity pathway (e.g., by targeting downstream signalling events). Different approaches are discussed below.

Inhibition of Obesity Expression or Obesity Activity

Any method that neutralizes or inhibits expression of the obesity gene (either transcription or translation), e.g. RORα1, RORα5 and RORα1-UNC5C, can be used to prevent or treat obesity.

For example, the administration of soluble peptides, proteins, fusion proteins, or antibodies (including anti-idiotypic antibodies) that bind to and “neutralize” circulating obesity can be used. Such obesity neutralizing peptides, proteins, fusion proteins, anti-idiotypic antibodies or Fabs are administered to a subject in amounts sufficient to treat or prevent obesity.

In an alternate embodiment, therapy can be designed to reduce the level of endogenous obesity gene expression, for example, using antisense or ribozyme approaches to inhibit or prevent translation of obesity mRNA-transcripts; triple helix approaches to inhibit transcription of the obesity gene; or targeted homologous recombination to inactivate or “knock out” the obesity gene or its endogenous promoter. Antisense, ribozyme or DNA constructs can be administered directly to the site containing the target cells; or can be directed to the target cells.

Antisense approaches involve the design of oligonucleotides (either DNA or RNA) that are complementary to obesity mRNA. The antisense oligonucleotides will bind to the complementary obesity mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, for example, the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. Thus, oligonucleotides complementary to either the 5′- or 3′-non-translated, non-coding regions of the obesity can be used in an antisense approach to inhibit translation of endogenous obesity mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of obesity mRNA, antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides, or at least 50 nucleotides in length.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

While antisense nucleotides complementary to the obesity coding region sequence could be used, those complementary to the transcribed untranslated region are most preferred. The antisense molecules should be delivered to cells which express the obesity in vivo. A number of methods have been developed for delivering antisense DNA or RNA to cells; for example, antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

Ribozyme molecules designed to catalytically cleave obesity mRNA transcripts can also be used to prevent translation of obesity mRNA and expression of obesity. (See, e.g., PCT International Publication WO90/11364, published Oct. 4, 1990; Sarver et al., 1990, Science 247:1222-1225). While ribozymes that cleave mRNA at site specific recognition sequences can be used to destroy obesity mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature 334:585-591. There are hundreds of potential hammerhead ribozyme cleavage sites within the nucleotide sequence of human obesity cDNA. Preferably the ribozyme is engineered so that the cleavage recognition site is located near the 5′ end of the OBESITY mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science 224:574-578; Zaug and Cech, 1986, Science 231:470-475; Zaug, et al., 1986, Nature 324:429-433; published International patent application No. WO 88/04300 by University Patents Inc.; Been and Cech, 1986, Cell 47:207-216). The Cech-type ribozymes have an eight basepair active site that hybridizes to a target RNA sequence, whereafter cleavage of the target RNA takes place. The invention encompasses those Cech-type ribozymes that target eight basepair active site sequences that are present in obesity.

As in the antisense approach, the ribozymes can be composed of modified oligonucleotides (e.g. for improved stability, targeting, etc.) and should be delivered to cells which express the obesity in vivo. A preferred method of delivery involves using a DNA construct “encoding” the ribozyme under the control of a strong constitutive pol III or pol II promoter, so that transfected cells will produce sufficient quantities of the ribozyme to destroy endogenous obesity messages and inhibit translation. Because ribozymes, unlike antisense molecules, are catalytic, a lower intracellular concentration is required for efficiency.

In an alternative embodiment for neutralizing circulating obesity, cells that are genetically engineered to express such soluble or secreted forms of obesity can be administered to a patient, whereupon they will serve as “bioreactors” in vivo to provide a continuous supply of the neutralizing protein.

Endogenous obesity gene expression can also be reduced by inactivating or “knocking out” the obesity gene or its promoter using targeted homologous recombination. (E.g., see Smithies et al., 1985, Nature 317:230-234; Thomas & Capecchi, 1987, Cell 51:503-512; Thompson et al., 1989, Cell 5:313-321; each of which is incorporated by reference herein in its entirety).

In yet another embodiment of the invention, the activity of obesity can be reduced using a “dominant negative” approach to effectuate obesity. To this end, constructs that encode defective obesity can be used in gene therapy approaches to diminish the activity of the obesity in appropriate target cells.

Pharmaceutical Composition Containing the Identified Candidate Compound

The compounds having the desired activity may be administered in a physiologically acceptable carrier to an obesity patient. Such compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal track, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on Formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound will naturally vary according to the nature and severity of the conditions, the age and sex of the animal or patient and the route of administration, according to well known principles of medicine.

In using a compound for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

It will be obvious to those skilled in the art to which the invention pertains, that various changes and modifications may be made without departing from the scope of the invention defined by the claims.

### EXAMPLES

### Example 1

Identification of Translocation Breakpoints

A male patient having a BMI of 48 was karyotyped and abnormal chromosomes were identified. It was found that the abnormal chromosomes had the translocation products between chromosome 4q22 and chromosome 15q22. The patient's sister and mother also had abnormally high BMI, and were also karyotyped (FIG. 1). It was subsequently found that the sister and mother were also carrying the balanced translocation. The breakpoints of each chromosome were further mapped with fish probes and southern blots and the breakpoints on each chromosome were sequenced. Using bacterial artificial chromosomes (BAC) clones spanning both breakpoints, the breakpoints were sequenced and assembled with the help of the public sequence to generate ordered contigs of the breakpoints region of chromosome 4 and 15. RORα1 on chromosome 15 was found to be affected since the breakpoint is within the first intron of isoform 1 resulting in the destruction of one copy of RORα1.

### Example 2

Gene Expression Analysis

Preadipocytes were isolated from the affected boy and girl and used to study adipocyte function. Gene expression was monitored for all genes mapped to the vicinity of the breakpoints both at the preadipocyte stage and after induction into mature adipocytes. The isolated adipocytes showed an altered phenotype compared with normal adipocytes both in general morphology, function and gene expression. Lipid droplets were incorporated as tiny droplets instead of the large ones normally seen. UNC5C was highly upregulated in the preadipocytes both before and after induction into mature adipocytes when compared with an adipocyte control cell line.

Two differently sized UNC5C transcripts could be detected in preadipocytes isolated from the patients. These were cloned and sequenced and shown to be a fusion between RORα1 exon 1 and UNC5C exon 2 giving rise to a novel protein. Both encoded a fusion transcript with the structure RORα1-UNC5C fusion (SEQ ID NO: 1 and SEQ ID NO: 3). The shorter of the two (SEQ ID NO: 3) is a splice version using alternative splice sites within exons 11 and 12 of UNC5C.

Patient Material

All three patients from the family were carriers of the translocation. The family consisted of affected mother and two affected siblings, the father is normal build. Lymphoblastoid cell lines were established according to standard procedures.

FISH Probes

For yeast artificial chromosome (YAC) screening, YACs from chromosome 4 and 15 from the CEPH human YAC library was used (YAC clones chromosome 4: 244C10, 676B9, 767G2, 783H11, 840F2, 854F4, 858A2, 871A1, 881A12 and chromosome 15: 667B2, 798C9, 801B4, 809G4, 827C7, 848E5, 854A11, 895G6, 897E9, 934G1, 937E4, 951F3). STS markers contained in the spanning YAC clones was used for PCR screening of bacterial artificial chromosome (BAC) libraries.

The CEPH Human BAC library was screened using STS markers D4S2407 and D4S1559 from YAC clone 858A2 on chromosome 4 and WI-7454 from YAC clone 798C9 on chromosome 15. STS marker AFMa244wc9 from chromosome 15 was used to screen BAC Human II library from Genome Systems, Inc.

Restriction digestion with BamHI and HindIII was used to assemble a contig of the BAC clones and flanking clones from each contig were selected for BAC end walk. BAC end sequencing was performed on whole BACs purified with Qiagen Large Construct Purification kit (Qiagen) using sp6 and T7 primers.

The end sequences of the BACs were used to screen the nr (non-redundant) and htgs (high throughput genomic sequences) databases at the NCBI/NIH server using the BLASTN program. Positive BAC hits were ordered from BACPAC Resources (Children's Hospital Oakland) and used as FISH probes.

To further map the breakpoints, sequences of approximately 6 kb from the spanning BACs RP11-402L11 (AC093832) and RP11-90A19 (AC079068) was derived by long range PCR and used as probes for FISH.

Mini FISH probes used for chromosome 4:

| Forward primer | Reverse primer |
|---|---|
| SEQ ID NO: 15 | SEQ ID NO: 16 |
| SEQ ID NO: 17 | SEQ ID NO: 18 |
| SEQ ID NO: 19 | SEQ ID NO: 20 |
| SEQ ID NO: 21 | SEQ ID NO: 22 |
| SEQ ID NO: 23 | SEQ ID NO: 24 |
| SEQ ID NO: 25 | SEQ ID NO: 26 |

Mini FISH probes used for chromosome 15:

| Forward primer | Reverse primer |
|---|---|
| SEQ ID NO: 27 | SEQ ID NO: 28 |
| SEQ ID NO: 29 | SEQ ID NO: 30 |
| SEQ ID NO: 31 | SEQ ID NO: 32 |
| SEQ ID NO: 33 | SEQ ID NO: 34 |
| SEQ ID NO: 35 | SEQ ID NO: 36 |
| SEQ ID NO: 37 | SEQ ID NO: 38 |
| SEQ ID NO: 39 | SEQ ID NO: 40 |
| SEQ ID NO: 41 | SEQ ID NO: 42 |
| SEQ ID NO: 43 | SEQ ID NO: 44 |

FISH Analysis

FISH was performed on metaphase chromosome spreads from the boy which is carrying the translocation. Metaphase spreads were prepared from lymphocytes according to standard methods.

Southern Blot

The probes used in southern blot were amplified by PCR from BAC clones RP11-402L11 (chromosome 4) and RP11-90A19 (chromosome 15) and labelled by random priming. The probes used for chromosome 4 were:

| Forward primer | Reverse primer |
|---|---|
| SEQ ID NO: 45 | SEQ ID NO: 46 |
| SEQ ID NO: 47 | SEQ ID NO: 48 |
| SEQ ID NO: 49 | SEQ ID NO: 50 |
| SEQ ID NO: 51 | SEQ ID NO: 52 |
| SEQ ID NO: 53 | SEQ ID NO: 54 |

The probes used for chromosome 15 were:

| Forward primer | Reverse primer |
|---|---|
| SEQ ID NO: 55 | SEQ ID NO: 56 |
| SEQ ID NO: 57 | SEQ ID NO: 58 |
| SEQ ID NO: 59 | SEQ ID NO: 60 |
| SEQ ID NO: 61 | SEQ ID NO: 62 |

PCR and Sequencing of the Breakpoints

The breakpoints could be bridged by long range PCR with chromosome derivate specific primers. For the chromosome 4 derivate the chromosome 4 primer SEQ ID NO: 63 and the chromosome 15 primer SEQ ID NO: 64 was used, and for the chromosome 15 derivate the chromosome 15 primer SEQ ID NO: 65 and the chromosome 4 primer SEQ ID NO: 66 was used.

The breakpoint on the chromosome 4 derivate PCR product could be sequenced using primer SEQ ID NO: 51 and the chromosome 15 derivate PCR product could be sequenced using primer SEQ ID NO: 52.

RNA Isolation

Total RNA was isolated from cells using Trizole reagent (Gibco) according to the manufacturer's description. The isolated RNA was dissolved in DEPC treated water and concentration was determined by spectrophotometric measurement at 260 nm. All samples were also electrophoresed in 1% agarose gel to assess integrity of ribosomal bands.

DNAse Treatment and cDNA Synthesis

15 μg of total RNA was treated with DNase to remove contaminating genomic DNA before cDNA synthesis. DNA-free™ from Ambion was used for this purpose. Shortly; 0.1 volume of 10 times DNase buffer, 6 units of DNase and water up to 30 μl was added to each sample. Samples were incubated at 37° for 40 minutes. DNase inactivation reagent was added to each sample and after a 2 minutes incubation at room temperature it was removed by centrifugation.

First strand cDNA synthesis was performed with SuperScript™First-Strand Synthesis System for RT-PCR (Invitrogen, Life Technonlogies) according to enclosed protocol. 2 μg of DNase treated total RNA was used in each synthesis. Priming of synthesis was accomplished with Oligo(dT)₁₂₋₁₈. For each synthesis reaction a negative control with no enzyme was set up.

PCR and Cloning

Full length cDNA of the fusion transcript was obtained with TaqPlus® Precision PCR System (Stratagene). In a total of 50 μl, 1 time TaqPlus Precision buffer, 200 μM of each dNTP (Applied Biosystems), 300 nM forward primer SEQ ID NO: 67, 300 nM reverse primer SEQ ID NO: 68 and 2.5U TaqPlus Precision polymerase mixture. cDNA from above was used as template, 0.4 μl/reaction (equivalent with 40 ng total RNA). The reaction mixture was then subjected to thermal cycling in an PTC-200 from MJ research. Thermal cycling included one step initial denaturation one minute at 95° C., 35 cycles with 95° C. one minute, 58° C. one minute, 72° C. three minutes and a final extension of ten minutes at 72°. The reaction mixture was then loaded on a 1% Low melt Agarose gel (BioRad). Inspection of the gel showed 2 bands of approximately 2.8 and 3 kb. Both bands were cut out and ligated into pcR2.1-TOPO® (Invitrogen) and sequentially transfected into TOP 10 (Invitrogen).

Both constructs were sequenced using Big Dye chemistry and 3100 Genetic Analyzer (Applied Biosystems).

Quantification

cDNA from above was also used for relative quantification on Applied Biosystems 7700.

Quantitative real-time PCR was carried out using SYBR® Green PCR Master Mix in 25 μL reactions run in triplicates. Template for the PCR was first strand cDNA from above (amount equivalent of 10 ng total RNA). Forward and reverse primers specific for UNC5c was used at a concentration of 400 nM. As negative control for amplification of remaining genomic DNA, no enzyme controls from cDNA synthesis was used.

Shotgun Library Construction

Human Bac clones where cultured over night in LB-media. DNA was prepared using Maxi-plasmid prep from Qiagen (VWR).

5 μg purified DNA was fragmented with a nebulizer.

DNA was fragmented to 1 to 3 kb fragments, checked on 1% agarose gel.

All according to the manufacturer's description in TOPO® Shotgun Sub cloning Kit (Invitrogen). DNA was Blunt-end repaired, dephosphorylated and transformed into One Shot competent E. coli, also from the kit

The transformation reaction was plated on LB plates and incubated over night. A colony picker Q-PIX (Genetix) picked the colonies into 96-well micro plates with LB-media

One more incubation 37° C. over night.

Hydra, a dispenser moved the bacterial suspension to 96-deep well plates and a 34-hour 37° C. shake incubation was performed. After centrifugation, continued with DNA preparation.

DNA Preparation

Add 50 μl water, vortex 15 minutes. When complete resuspension add 70 μl Stet-Tween20 with 5.2 mg/ml Rnase and 8.4 mg/ml Lysozyme. Denaturate in microwave 1 minute add 335 μl water, vortex and let stand in ice for 10 minutes. Spin boxes for 30 minutes×4000 rpm. The clear supernatant moves to new 96-well micro plates to be sequenced.

Sequencing

Big Dye chemistry primer T3, primer T7 and ABI PRISM 3700 DNA Analyzer (Applied Biosystems) was used for sequencing clones.

2 × 96 plate

| | |
|---|---|
| Big Dye mix | 220 μl (v.2.0) |
| Water | 220 μl |
| Buffer × 2, 5 | 660 μl (200 mMTris-HCl ph 9.0, 5 mM MgCl2) |
| Primer T7 or T3 | 7 μl (100 pmol/μl) |

Robot Biomek 2000, loaded the mix and the DNA samples into PCR-plates.
PTC-200, (MJ Research) the thermal cycling instrument;
95° C.×2′, (95° C.×10″, 50° C.×10″, 60° C.×4′)×35 cycler and 8° C.
Followed by centrifugation and ethanol cleaning up step.
15 μl water was added before running on 3700.
5′ Race of RORα
Template used was Marathon Ready cDNA from Clontech (Human Adipocyte #7447(pooled from 11 male/female Caucasians, ages 19-57).
Nested PCR was used to obtain discrete bands from the PCR reactions. In both reactions following concentrations was used; forward and reverse primer 200 nM, dNTP 200 μM, MgCl 2 mM, 0.125U/μL AmpliTaq Gold (Applied Biosystems) all in one time PCR buffer II (Applied Biosystems).
In the first round, forward primer was AP1 (linker primer, Clontech) (SEQ ID NO: 69). Reverse primer, situated in the RORalpha sequence (SEQ ID NO: 70). Template 2 μL (˜0.1 ng/μL, Marathon Ready cDNA). This reaction mixture was then subjected to thermal cycling in a PTC-200 from MJ research. Thermal cycling included one step initial denaturation of eight minutes at 95° C., 30 cycles with 94° C. fortyfive seconds, 66° C. fortyfive seconds, 72° C. one minute and a final extension of ten minutes at 72°.
In the second round of PCR, forward primer was AP2 (linker primer, Clontech) (SEQ ID NO: 71). Reverse primer was the same as was used in the first round of PCR (SEQ ID NO: 70). Temlate was 1 μL of a twenty times dilution of the first PCR-reaction. This mixture was subjected to another 30 cycles, as above, but with 64 instead of 66° C. as annealing temperature.

PCR reactions were loaded on a low melt agarose gel (1% from BioRad). Potentially interesting bands were chosen and cut out from the gel, slices melted at 65° and ligated into pcR2.1-TOPO® (Invitrogen). Ligation mixture was then used to transfect TOP 10 (Invitrogen).

Two clones from each band was then sequenced using Big Dye chemistry and 3100 Genetic Analyzer (Applied Biosystems).

Taqman Analysis of RORα5

Primers where designed with Applied Biosystems program Primer Express 2.0 Forward primer Tm 58.5° C. was SEQ ID NO: 72. Reverse primer Tm 58.5° C. was SEQ ID NO: 73. Template poly A+ RNA from the following human tissues was obtained from Clontech; Bone marrow, brain, kidney, liver, lung, skeletal muscle, small intestine, spleen, testis and fat. cDNA synthesis was made with SuperScript™ First-Strand Synthesis System for RT-PCR, Invitrogen™ according to the protocol supplied by the manufacturer. Real time PCR was performed according to protocol from the manufacturer and run on ABI Prism 7700® Sequence Detection System, Applied Biosystems. Results analysed with Sequence detector V1.7

PCR Detection of UNC5C Splice Variant Transcript

Forward primer was SEQ ID NO: 74 spanning bases 1586-1604 of human UNC5C sequence AF055634. Reverse primer was SEQ ID NO 75 spanning 2386-2367 of transcript AF055634. Template was cDNA from human brain (mRNA from Clontech converted into cDNA using “Superscript™” First-Strand Synthesis System for RT-PCR” (Invitrogen)) according to protocol from the manufacturer. This PCR resulted in 3 bands. The expected one at ˜800 bp, a very faint one that was slightly shorter and one at ˜550 bp. The 800 and 550 bands were cut out of the gel and purified using Qiaquick gel extraction kit (Qiagen) and then sequenced using Big Dye chemistry and 3100 Genetic Analyzer (Applied Biosystems). The 800 band was the expected UNC5c sequence i.e. partial UNC5c spanning bases 1586-2386. The 550 bp fragment was UNC5C with a deletion of bases 1873-2078. This deletion is identical to the deletion in the sequence of UNC5Cb with accession number AAC90914.

Bioinformatics

Sequence assembly and alignment of gene sequences was made using Sequencer software from Gene codes corporation version 4.1.2 on a Macintosh G4.

Primary Cell Culture

The following products were purchased from Biohittaker (MD, USA): DMEM F-12, FBM (fibroblast basal medium), human fibroblast growth factor-β (hFGF-β; 1 μg/ml), Trypsin EDTA (200 mg/ml and 500 mg/ml), Fungizone (250 μg/ml). Foetal calf serum (FCS) was provided from Harlan Sera lab (Leicestershire, UK). Pioglitasone (X-46) was from AstraZeneca R&D Molndal.

0.008M Phosphate buffered saline (PBS); insulin and transferrin were obtained from Life Technologies. BSA fraction V, Collagenase (C-6885), IBMX (3-isobutyl-1-methylxanthine), dexamethasone, 0.05M Tris Buffered Saline pH 8.0 were purchased from SIGMA. β-mercaptoethanol and glucose were from Kebo lab. Primaria™ tissue culture flasks were from Falcon. Nile Red was from Molecular Probes.

Biopsies from humans were taken under aseptic conditions and transferred to DMEM/F12 supplemented with 25 μg/ml Gentamycin and Fungizone (2.5 μg/ml). The adipose tissue was digested in DMEM F-12 containing albumin (1 mg/ml), collagenase (1 mg/ml), glucose (2 mg/ml), 25 μg/ml Gentamycin and Fungizone (2.5 μg/ml) in a shaking waterbath at 37° C. for 30-45 min. The cell suspension was then filtered through a nylon mesh (100 μm) and centrifuged at 650 g for 10 minutes. The sedimented cells, preadipocytes, were resuspended and washed twice in DMEM F-12 supplemented with 25 μg/ml Gentamycin and Fungizone (2.5 μg/ml). After the last wash the cells were resuspended in FBM (fibroblast medium) supplemented with 10% FCS, 25 μg/ml Gentamycin and Fungizone (2.5 μg/ml, 0.1% 0.1M mercaptoethanol, 10 mg/ml insulin, 10 mg/ml transferrin and 0.001 μg/ml hFGF-β The cells was seeded and incubated in small Primaria culture flasks (25 cm²) over night followed by washing with PBS and culture continued until the cells reached confluence.

Thereafter the cells were washed in PBS (without Mg⁺⁺ and Ca⁺⁺), treated with trypsin EDTA for a few minutes, resuspended and splited in fresh FBM and seeded into new small culture flasks. The medium was changed every day. The new cell lines were named PSc020108 (male) and Asc020108 (female).

The fraction with the mature adipocytes (after protease treatment) was frozen in −80° C. for further RNA preparation.

Differentiation

The undifferentiated preadipocytes were seeded into small cell culture flasks and cultured in DMEM/F12 supplemented with 10% FCS, 1% PEST, 10 μg/ml insulin, 10 μg/ml transferrin and 0.1 mM β-mercaptoethanol (referred to as standard medium). When the cells have reached confluence the differentiation was induced by adding standard medium supplemented with 0.25 μM dexamethasone and 0.5 mM IBMX (referred to as induction medium 1) for three days. To maintain and extend the differentiation process the medium was changed to standard medium containing 10 μM X-46 and 0.25 μM Dexamethasone (referred to as induction medium 2) for 2 days. This process was repeated once and after that the culture process was continued only with standard medium supplemented with 0.25 μM dexamethasone (referred to as maintenance medium 1).

The lipid accumulation in the adipocytes was confirmed by Nile Red staining.

Staining Cells with Nile Red

Nile red is a benzophenoxazone dye. In older chemical and histochemical literature the dye sometimes referred to as nileblue A-oxazone. Nile red is poorly soluble in water but does dissolve in a wide variety of organic solvents.

A 1 mg/ml stock solution of Nile red in acetone were prepared and stored protected from light. Staining could be carried out on either fixed (1.5% glutaraldehyde in PBS, for 5 min) or unfixed cells. Attached unfixed cultured cells were covered with PBS. The dye was then added directly to the flask to effect a 1:100 dilution to yield a final concentration of 10 μg/ml. The preparation was incubated for a minimum of 5 min. For fluorescence microscopy excess dye was removed by briefly rinsing the cells with PBS. The suspension medium must not contain serum or albumin since they could act as a sink to draw Nile red out of the cells. Dye in the medium does not interfere with the observations since Nile red fluorescence is quenched in water. Nile red is not toxic for the cells so it is possible to continue the culture after staining when unfixed cells are used.

Morphology and Fluorescence Microscopy

The differentiation of adipocyte precursors into mature adipocytes was assessed by conventional microscopy at a 100-200-fold magnification. Cells were considered differentiated when their cytoplasm was filled with lipid droplets, clearly seen in phase contrast. The fluorescence microscopy studies was carried out using a Zeiss axiovert 25 inverted microscope, equipped with a HBO 50 illuminator and with a CP-Achromat 10×/0.25 ph1/Var1 objective lens and the excitation wavelength was 485 nm (filter set 487909).

The stained cells were photographed in color using the AxioVision system from Carl Zeiss. The pictures were processed in Adobe PhotoShop 6.0.

1. An isolated nucleic acid molecule comprising a nucleotide sequence having at least 65% identity to a degenerate variant of SEQ ID NO: 1, SEQ ID NO: 3 or SEQ ID NO: 7.

2. The isolated nucleic acid molecule of claim 1, wherein the nucleic acid molecule comprises the nucleotide sequence of SEQ ID NO: 1, SEQ ID NO: 3 or SEQ ID NO: 7.

3. An isolated nucleic acid molecule comprising the complement of SEQ ID NO: 1, SEQ ID NO: 3 or SEQ ID NO:7.

4. An isolated nucleic acid molecule encoding a RORα1-UNC5C polypeptide, said nucleic acid molecule comprising a nucleotide sequence having at least 65% identity to a degenerate variant of SEQ ID NO: 1 or SEQ ID NO: 3.

5. An isolated nucleic acid molecule encoding a RORα5 polypeptide, said nucleic acid molecule comprising a nucleotide sequence having at least 65% identity to a degenerate variant of SEQ ID NO: 7.

6. A vector comprising the nucleic acid molecule of any one of claims 1-5.

7. A host cell comprising the vector of claim 6.

8. A purified fusion polypeptide of the RORα1-UNC5C polypeptide comprising an amino acid sequence having at least 65% identity to the amino acid sequence of SEQ ID NO: 2 or SEQ ID NO: 4.

9. The purified polypeptide of claim 8, wherein the amino acid sequence comprises the amino acid sequence of SEQ ID NO: 2 or SEQ ID NO:4.

10. A purified polypeptide of the RORα5 polypeptide comprising an amino acid sequence having at least 65% identity to the amino acid sequence of SEQ ID NO: 8.

11. The purified polypeptide of claim 10, wherein the amino acid sequence comprises the amino acid sequence of SEQ ID NO: 8.

12. A method for producing a protein comprising:
a) culturing the host cell of claim 7 under conditions whereby the protein is produced, and
b) recovering the protein from the host cell culture.

13. A method for detecting a polynucleotide which encodes a protein comprising the amino acid sequence of SEQ ID NO:2, SEQ ID NO:4 or SEQ ID NO: 8 in a biological sample comprising the steps of:
a) hybridizing a complement of the polynucleotide sequence which encodes SEQ ID NO: 2, SEQ ID NO:4 or SEQ ID NO: 8 to a nucleic acid material of a biological sample, thereby forming a hybridization complex; and
b) detecting the hybridization complex, wherein the presence of the complex correlates with the presence of a polynucleotide encoding a protein in the biological sample.

14. A method for detecting the presence of an obesity susceptibility gene comprising:
determining the identity of a gene located 20 cM, or less, adjacent to the translocation breakpoint between chromosome 4 at cytoband 4q22.3 and chromosome 15 at cytoband 15q22.2; and
determining if the gene has aberrant gene expression as a consequence of the translocation event.

15. A method for detecting the presence of a translocation junction between chromosome 4 at cytoband 4q22.3 and chromosome 15 at cytoband 15q22.2 comprising analysing a sample of DNA from an individual for the presence of the translocation junction.

16. A method for identifying a test compound that modulates the expression of an obesity susceptibility gene identified in claim 14 comprising:
contacting a cell capable of expressing the susceptibility gene with a test compound; and
determining the level of expression of the obesity susceptibility gene in the presence of the test compound, wherein a decrease or an increase in expression of a obesity susceptibility gene, as compared to the level of expression of an obesity susceptibility gene in the absence of the compound, is indicative that the test compound modulates the expression of the obesity susceptibility gene.

17. A method of identifying a test compound that modulates the activity of an obesity protein encoded by the obesity susceptibility gene identified in claim 14, comprising:
contacting the obesity protein with a test compound; and
determining the level of activity of the obesity protein in the presence of the compound, wherein a decrease or an increase in obesity protein activity, as compared to the level of activity of the obesity protein in the absence of the compound, is indicative that the test compound modulates obesity protein activity.

18. A method of treating a subject having obesity comprising administering an effective amount of the compound identified in any one of claims 16 or 17.

19. (canceled)

20. A pharmaceutical composition comprising a compound identified in any one of claims 16 or 17, and a pharmaceutically acceptable adjuvant, diluent or carrier.

21. A method of making a pharmaceutical composition comprising:
contacting a cell capable of expressing an obesity susceptibility gene with a test compound;
determining the level of expression of the obesity susceptibility gene in the presence of the test compound, wherein a decrease in expression of a obesity susceptibility gene, as compared to the level of expression of a obesity susceptibility gene in the absence of the compound, is indicative that the test compound decreases obesity susceptibility gene expression; and
formulating the test compound that decreases obesity susceptibility gene expression into a pharmaceutical composition.

22. A method of making a pharmaceutical composition comprising:
contacting an obesity protein with a test compound;
determining the level of activity of the obesity susceptibility protein in the presence of the compound, wherein a decrease in obesity protein activity, as compared to the level of activity of the obesity protein in the absence of the compound, is indicative that the test compound decreases obesity protein activity; and
formulating the test compound that decreases obesity protein activity into a pharmaceutical composition.

23. A method for determining if an obesity susceptibility gene identified in claim 14 has an altered level of gene expression comprising:
comparing the level of obesity gene expression in a cell from a patient having obesity with a control cell, and
determining the level of expression of the obesity susceptibility gene in both cells, wherein a decrease or an increase in expression of the obesity susceptibility gene, as compared to the level of expression of the obesity susceptibility gene in the control cell, indicates that the obesity susceptibility gene has altered gene expression.

24. A method of diagnosing obesity, or a susceptibility thereto in a subject, the method comprising:
determining the level of mRNA of UNC5C; and
comparing the level of mRNA of UNC5C in the sample with a control, wherein an increase in the level of UNC5C in the sample compared to the control indicates that the subject has obesity, or a susceptibility thereto.

25. A method of diagnosing obesity or a susceptibility thereto in a subject, the method comprising:
determining the level of aUNC5C protein in a sample from a subject; and
comparing the level of UNC5C in the sample with a control, wherein an increase in the level of the protein in the sample compared to the control indicates that the subject has obesity, or a susceptibility thereto.

26. A method of diagnosing obesity, or a susceptibility thereto in a subject, the method comprising analysing for the presence of ROR1α1-UNC5C mRNA, wherein the presence of the mRNA indicates that the subject has obesity, or a susceptibility thereto.

27. A method of diagnosing obesity or a susceptibility thereto in a subject, the method comprising analysing for the presence of the ROR1α1-UNC5C fusion polypeptide, wherein the presence of the polypeptide indicates that the subject has obesity, or a susceptibility thereto.


SEQUENCE LISTING (75 sequences)

SEQ ID NO: 1 - DNA, 2986 bp, Homo sapiens
ctgaaaacagaagatagagaggagtctcggagctcgccatctccagcgatctctacattgggaaaaaacatggagtcagctccggcagcccccgaccccgccgccagcgagccaggcagcagcggcgcggacgcggccgccggctccagggagaccccgctgaaccaggaatccgcccgcaagagcgagccgcctgccccggtgcgcagacagagctattccagcaccagcagagatgatgacttttttcatgaactcccagaaacttttccttctgatccacctgagcctctgccacatttccttattgagcctgaagaagcttatattgtgaagaataagcctgtgaacctgtactgtaaagcaagccctgccacccagatctatttcaagtgtaatagtgaatgggttcatcagaaggaccacatagtagatgaaagagtagatgaaacttccggtctcattgtccgggaagtgagcattgagatttcgcgccagcaagtggaagaactctttggacctgaagattactggtgccagtgtgtggcctggagctccgcgggtaccacaaagagccggaaggcgtatgtgcgcattgcatatctacggaagacatttgagcaggaacccctaggaaaggaagtgtctttggaacaggaagtcttactccagtgtcgaccacctgaagggatcccagtggctgaggtggaatggttgaaaaatgaagacataattgatcccgttgaagatcggaatttttatattactattgatcacaacctcatcataaagcaggcccgactctctgatactgcaaattacacctgtgttgccaaaaacattgttgccaagaggaaaagtacaactgccactgtcatagtctatgtcaacggtggctggtccacctggacggagtggtctgtgtgtaacagccgctgtggacgagggtatcagaaacgtacaaggacttgtaccaacccggcaccactcaatgggggtgccttctgtgaagggcagagtgtgcagaaaatagcctgtactacgttatgcccagtggatggcaggtggacgccatggagcaagtggtctacttgtggaactgagtgcacccactggcgcaggagggagtgcacggcgccagcccccaagaatggaggcaaggactgcgacggcctcgtcttgcaatccaagaactgcactgatgggctttgcatgcagagtttcatttatcccatttcaactgaacagagaacccagaatgaatatggattttcttctgctcctgattcagatgatgttgctctctatgttgggattgtgatagcagtgatcgtttgcctggcgatctctgtagttgtggccttgtttgtgtatcggaagaatcatcgtgactttgagtcagatattattgactcttcggcactcaatgggggctttcagcctgtgaacatcaaggcagcaagacaagatctgctggctgtacccccagacctcacgtcagctgcagccatgtacagaggacctgtctatgccctgcatgacgtctcagacaaaatcccaatgaccaactctccaattctggatccactgcccaacctgaaaatcaaagtgtacaacacctcaggtgctgtcaccccccaagatgacctctctgagtttacgtccaagctgtcccctcagatgacccagtcgttgttggagaatgaagccctcagcctgaagaaccagagtctagcaaggcagactgatccatcctgtaccgcatttggcagcttcaactcactgggaggtcaccttattgttcccaattcaggagtcagcttgctgattcccgctggggccattccccaagggagagtctacgaaatgtatgtgactgtacacaggaaagaaactatgaggccacccatggatgactctcagacacttttgacccctgtggtgagctgtgggcccccaggagctctgctcacccgccccgtcgtcctcactatgcatcactgcgcagaccccaataccgaggactggaaaatactgctcaagaaccaggcagcacagggacagtgggaggatgtggtggtggtcggggaggaaaacttcaccaccccctgctacattcagctggatgcagaggcctgccacatcctcacagagaacctcagcacctacgccctggtaggacattccaccaccaaagcggctgcaaagcgcctcaagctggccatctttgggcccctgtgctgctcctcgctggagtacagcatccgagtctactgtctggatgacacccaggatgccctgaaggaaattttacatcttgagagacagacgggaggacagctcctagaagaacctaaggctcttcattttaaaggcagcacccacaacctgcgcctgtcaattcacgatatcgcccattccctctggaagagcaaattgctggctaaatatcaggaaattccattttaccatgtttggagtggatctcaaagaaacctgcactgcaccttcactctggaaagatttagcctgaacacagtggagctggtttgcaaactctgtgtgcggcaggtggaaggagaagggcagatcttccagctcaactgcaccgtgtcagaggaacctactggcatcgatttgccgctgctggatcctgcgaacaccatcaccacggtcacggggcccagtgctttcagcatccctctccctatccggcagaagctctgtagcagcctggatgccccccagacgagaggccatgactggaggatgctggcccataagctgaacctggacaggtacttgaattactttgccaccaaatccagcccaactggcgtaatcctggatctttgggaagcacagaacttcccagatggaaacctgagcatgctggcagctgtcttggaagaaatgggaagacatgaaacggtggtgtccttagcagcagaagggcagtattaaccaccatgctggaaggggaaat

SEQ ID NO: 2 - protein, 964 aa, Homo sapiens
MESAPAAPDPAASEPGSSGADAAAGSRETPLNQESARKSEPPAPVRRQSYSSTSRDDDFFHELPETFPSDPPEPLPHFLIEPEEAYIVKNKPVNLYCKASPATQIYFKCNSEWVHQKDHIVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPVEDRNFYITIDHNLIIKQARLSDTANYTCVAKNIVAKRKSTTATVIVYVNGGWSTWTEWSVCNSRCGRGYQKRTRTCTNPAPLNGGAFCEGQSVQKIACTTLCPVDGRWTPWSKWSTCGTECTHWRRRECTAPAPKNGGKDCDGLVLQSKNCTDGLCMQSFIYPISTEQRTQNEYGFSSAPDSDDVALYVGIVIAVIVCLAISVVVALFVYRKNHRDFESDIIDSSALNGGFQPVNIKAARQDLLAVPPDLTSAAAMYRGPVYALHDVSDKIPMTNSPILDPLPNLKIKVYNTSGAVTPQDDLSEFTSKLSPQMTQSLLENEALSLKNQSLARQTDPSCTAFGSFNSLGGHLIVPNSGVSLLIPAGAIPQGRVYEMYVTVHRKETMRPPMDDSQTLLTPVVSCGPPGALLTRPVVLTMHHCADPNTEDWKILLKNQAAQGQWEDVVVVGEENFTTPCYIQLDAEACHILTENLSTYALVGHSTTKAAAKRLKLAIFGPLCCSSLEYSIRVYCLDDTQDALKEILHLERQTGGQLLEEPKALHFKGSTHNLRLSIHDIAHSLWKSKLLAKYQEIPFYHVWSGSQRNLHCTFTLERFSLNTVELVCKLCVRQVEGEGQIFQLNCTVSEEPTGIDLPLLDPANTITTVTGPSAFSIPLPIRQKLCSSLDAPQTRGHDWRMLAHKLNLDRYLNYFATKSSPTGVILDLWEAQNFPDGNLSMLAAVLEEMGRHETVVSLAAEGQY

SEQ ID NO: 3 - DNA, 2780 bp, Homo sapiens
ctgaaaacagaagatagagaggagtctcggagctcgccatctccagcgatctctacattgggaaaaaacatggagtcagctccggcagcccccgaccccgccgccagcgagccaggcagcagcggcgcggacgcggccgccggctccagggagaccccgctgaaccaggaatccgcccgcaagagcgagccgcctgccccggtgcgcagacagagctattccagcaccagcagagatgatgacttttttcatgaactcccagaaacttttccttctgatccacctgagcctctgccacatttccttattgagcctgaagaagcttatattgtgaagaataagcctgtgaacctgtactgtaaagcaagccctgccacccagatctatttcaagtgtaatagtgaatgggttcatcagaaggaccacatagtagatgaaagagtagatgaaacttccggtctcattgtccgggaagtgagcattgagatttcgcgccagcaagtggaagaactctttggacctgaagattactggtgccagtgtgtggcctggagctccgcgggtaccacaaagagccggaaggcgtatgtgcgcattgcatatctacggaagacatttgagcaggaacccctaggaaaggaagtgtctttggaacaggaagtcttactccagtgtcgaccacctgaagggatcccagtggctgaggtggaatggttgaaaaatgaagacataattgatcccgttgaagatcggaatttttatattactattgatcacaacctcatcataaagcaggcccgactctctgatactgcaaattacacctgtgttgccaaaaacattgttgccaagaggaaaagtacaactgccactgtcatagtctatgtcaacggtggctggtccacctggacggagtggtctgtgtgtaacagccgctgtggacgagggtatcagaaacgtacaaggacttgtaccaacccggcaccactcaatgggggtgccttctgtgaagggcagagtgtgcagaaaatagcctgtactacgttatgcccagtggatggcaggtggacgccatggagcaagtggtctacttgtggaactgagtgcacccactggcgcaggagggagtgcacggcgccagcccccaagaatggaggcaaggactgcgacggcctcgtcttgcaatccaagaactgcactgatgggctttgcatgcagagtttcatttatcccatttcaactgaacagagaacccagaatgaatatggattttcttctgctcctgattcagatgatgttgctctctatgttgggattgtgatagcagtgatcgtttgcctggcgatctctgtagttgtggccttgtttgtgtatcggaagaatcatcgtgactttgagtcagatattattgactcttcggcactcaatgggggctttcagcctgtgaacatcaaggcagcaagacaagatctgctggctgtacccccagacctcacgtcagctgcagccatgtacagaggacctgtctatgccctgcatgacgtctcagacaaaatcccaatgaccaactctccaattctggatccactgcccaacctgaaaatcaaagtgtacaacacctcaggtgctgtcaccccccaagatgacctctctgagtttacgtccaagctgtcccctcagatgacccagtcgttgttggagaatgaagccctcagcctgaagaaccagagtctagcaaggcagactgatccatcctgtaccgcatttggcagcttcaactcactgggaggtcaccttattgttcccaattcaggagtcagcttgctgattcccgctggggccattccccaagggagagtctacgaaatgtatgtgactgtacacaggaaagaaactatgaggccacccatggatgactctcagacacttttgacccctgtggtgagctgtgggcccccaggagctctgctcacccgccccgtcgtcctcactatgcatcactgcgcagaccccaataccgaggactggaaaatactgctcctcgctggagtacagcatccgagtctactgtctggatgacacccaggatgccctgaaggaaattttacatcttgagagacagacgggaggacagctcctagaagaacctaaggctcttcattttaaaggcagcacccacaacctgcgcctgtcaattcacgatatcgcccattccctctggaagagcaaattgctggctaaatatcaggaaattccattttaccatgtttggagtggatctcaaagaaacctgcactgcaccttcactctggaaagatttagcctgaacacagtggagctggtttgcaaactctgtgtgcggcaggtggaaggagaagggcagatcttccagctcaactgcaccgtgtcagaggaacctactggcatcgatttgccgctgctggatcctgcgaacaccatcaccacggtcacggggcccagtgctttcagcatccctctccctatccggcagaagctctgtagcagcctggatgccccccagacgagaggccatgactggaggatgctggcccataagctgaacctggacaggtacttgaattactttgccaccaaatccagcccaactggcgtaatcctggatctttgggaagcacagaacttcccagatggaaacctgagcatgctggcagctgtcttggaagaaatgggaagacatgaaacggtggtgtccttagcagcagaagggcagtattaaccaccatgctggaaggggaaat

SEQ ID NO: 4 - protein, 669 aa, Homo sapiens
MESAPAAPDPAASEPGSSGADAAAGSRETPLNQESARKSEPPAPVRRQSYSSTSRDDDFFHELPETFPSDPPEPLPHFLIEPEEAYIVKNKPVNLYCKASPATQIYFKCNSEWVHQKDHIVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPVEDRNFYITIDHNLIIKQARLSDTANYTCVAKNIVAKRKSTTATVIVYVNGGWSTWTEWSVCNSRCGRGYQKRTRTCTNPAPLNGGAFCEGQSVQKIACTTLCPVDGRWTPWSKWSTCGTECTHWRRRECTAPAPKNGGKDCDGLVLQSKNCTDGLCMQSFIYPISTEQRTQNEYGFSSAPDSDDVALYVGIVIAVIVCLAISVVVALFVYRKNHRDFESDIIDSSALNGGFQPVNIKAARQDLLAVPPDLTSAAAMYRGPVYALHDVSDKIPMTNSPILDPLPNLKIKVYNTSGAVTPQDDLSEFTSKLSPQMTQSLLENEALSLKNQSLARQTDPSCTAFGSFNSLGGHLIVPNSGVSLLIPAGAIPQGRVYEMYVTVHRKETMRPPMDDSQTLLTPVVSCGPPGALLTRPVVLTMHHCADPNTEDWKILLLAGVQHPSLLSG

SEQ ID NO: 5 - DNA, 1863 bp, Homo sapiens
gttttttttttttttttggtaccatagagttgctctgaaaacagaagatagagggagtctcggagctcgcatctccagcgatctctacattgggaaaaaacatggagtcagctccggcagcccccgaccccgccgccagcgagccaggcagcagcggcgcggacgcggccgccggctccagggagaccccgctgaaccaggaatccgcccgcaagagcgagccgcctgccccggtgcgcagacagagctattccagcaccagcagaggtatctcagtaacgaagaagacacatacatctcaaattgaaattattccatgcaagatctgtggagacaaatcatcaggaatccattatggtgtcattacatgtgaaggctgcaagggctttttcaggagaagtcagcaaagcaatgccacctactcctgtcctcgtcagaagaactgtttgattgatcgaaccagtagaaaccgctgccaacactgtcgattacagaaatgccttgccgtagggatgtctcgagatgctgtaaaatttggccgaatgtcaaaaaagcagagagacagcttgtatgcagaagtacagaaacaccggatgcagcagcagcagcgcgaccaccagcagcagcctggagaggctgagccgctgacgcccacctacaacatctcggccaacgggctgacggaacttcacgacgacctcagtaactacattgacgggcacacccctgaggggagtaaggcagactccgccgtcagcagcttctacctggacatacagccttccccagaccagtcaggtcttgatatcaatggaatcaaaccagaaccaatatgtgactacacaccagcatcaggcttctttccctactgttcgttcaccaacggcgagacttccccaactgtgtccatggcagaattagaacaccttgcacagaatatatctaaatcgcatctggaaacctgccaatacttgagagaagagctccagcagataacgtggcagacctttttacaggaagaaattgagaactatcaaaacaagcagcgggaggtgatgtggcaattgtgtgccatcaaaattacagaagctatacagtatgtggtggagtttgccaaacgcattgatggatttatggaactgtgtcaaaatgatcaaattgtgcttctaaaagcaggttctctagaggtggtgtttatcagaatgtgccgtgcctttgactctcagaacaacaccgtgtactttgatgggaagtatgccagccccgacgtcttcaaatccttaggttgtgaagactttattagctttgtgtttgaatttggaaagagtttatgttctatgcacctgactgaagatgaaattgcattattttctgcatttgtactgatgtcagcagatcgctcatggctgcaagaaaaggtaaaaattgaaaaactgcaacagaaaattcagctagctcttcaacacgtcctacagaagaatcaccgagaagatggaatactaacaaagttaatatgcaaggtgtctacattaagagccttatgtggacgacatacagaaaagctaatggcatttaaagcaatatacccagacattgtgcgacttcattttcctccattatacaaggagttgttcacttcagaatttgagccagcaatgcaaattgatgggtaaatgttatcacctaagcacttctagaatgtctgaagtacaaacatgaaaaacaaacaaaaaaattaaccgagacactttatatggccctgcacagacctggagcgccacacactgcacatcttttggtgatcggggtcaggcaaaggaggggaaacaatgaaaacaaataaagttgaacttgtttttctca

SEQ ID NO: 6 - protein, 523 aa, Homo sapiens
MESAPAAPDPAASEPGSSGADAAAGSRETPLNQESARKSEPPAPVRRQSYSSTSRGISVTKKTHTSQIEIIPCKICGDKSSGIHYGVITCEGCKGFFRRSQQSNATYSCPRQKNCLIDRTSRNRCQHCRLQKCLAVGMSRDAVKFGRMSKKQRDSLYAEVQKHRMQQQQRDHQQQPGEAEPLTPTYNISANGLTELHDDLSNYIDGHTPEGSKADSAVSSFYLDIQPSPDQSGLDINGIKPEPICDYTPASGFFPYCSFTNGETSPTVSMAELEHLAQNISKSHLETCQYLREELQQITWQTFLQEEIENYQNKQREVMWQLCAIKITEAIQYVVEFAKRIDGFMELCQNDQIVLLKAGSLEVVFIRMCRAFDSQNNTVYFDGKYASPDVFKSLGCEDFISFVFEFGKSLCSMHLTEDEIALFSAFVLMSADRSWLQEKVKIEKLQQKIQLALQHVLQKNHREDGILTKLICKVSTLRALCGRHTEKLMAFKAIYPDIVRLHFPPLYKELFTSEFEPAMQIDG

SEQ ID NO: 7 - DNA, 1821 bp, Homo sapiens
ctagattcctctgtgtttcatacccaggaggtcagggagggcaccttgtagtgcctgaggccccgagagggttcacagccacctgagaagtgtttgcagtctatgggtgtgtgttctttctccaagctgtgtcagctgtgggatggcgtggagagaaaacaagaaaaagttgtttccctcagctctctgctgaaaaaatgctcacggggagtcagtcaacatttggtatctcagtaacgaagaagacacatacatctcaaattgaaattattccatgcaagatctgtggagacaaatcatcaggaatccattatggtgtcattacatgtgaaggctgcaagggctttttcaggagaagtcagcaaagcaatgccacctactcctgtcctcgtcagaagaactgtttgattgatcgaaccagtagaaaccgctgccaacactgtcgattacagaaatgccttgccgtagggatgtctcgagatgctgtaaaatttggccgaatgtcaaaaaagcagagagacagcttgtatgcagaagtacagaaacaccggatgcagcagcagcagcgcgaccaccagcagcagcctggagaggctgagccgctgacgcccacctacaacatctcggccaacgggctgacggaacttcacgacgacctcagtaactacattgacgggcacacccctgaggggagtaaggcagactccgccgtcagcagcttctacctggacatacagccttccccagaccagtcaggtcttgatatcaatggaatcaaaccagaaccaatatgtgactacacaccagcatcaggcttctttccctactgttcgttcaccaacggcgagacttccccaactgtgtccatggcagaattagaacaccttgcacagaatatatctaaatcgcatctggaaacctgccaatacttgagagaagagctccagcagataacgtggcagacctttttacaggaagaaattgagaactatcaaaacaagcagcgggaggtgatgtggcaattgtgtgccatcaaaattacagaagctatacagtatgtggtggagtttgccaaacgcattgatggatttatggaactgtgtcaaaatgatcaaattgtgcttctaaaagcaggttctctagaggtggtgtttatcagaatgtgccgtgcctttgactctcagaacaacaccgtgtactttgatgggaagtatgccagccccgacgtcttcaaatccttaggttgtgaagactttattagctttgtgtttgaatttggaaagagtttatgttctatgcacctgactgaagatgaaattgcattattttctgcatttgtactgatgtcagcagatcgctcatggctgcaagaaaaggtaaaaattgaaaaactgcaacagaaaattcagctagctcttcaacacgtcctacagaagaatcaccgagaagatggaatactaacaaagttaatatgcaaggtgtctacattaagagccttatgtggacgacatacagaaaagctaatggcatttaaagcaatatacccagacattgtgcgacttcattttcctccattatacaaggagttgttcacttcagaatttgagccagcaatgcaaattgatgggtaaatgttatcacctaagcacttctagaatgtctgaagtacaaacatgaaaaacaaacaaaaaaattaaccgagacactttatatggccctgcacagacctggagcgccacacactgcacatcttttggtgatcggggtcaggcaaaggaggggaaacaatgaaaacaaataaagttgaacttgtttttctca

SEQ ID NO: 8 - protein, 477 aa, Homo sapiens
MLTGSQSTFGISVTKKTHTSQIEIIPCKICGDKSSGIHYGVITCEGCKGFFRRSQQSNATYSCPRQKNCLIDRTSRNRCQHCRLQKCLAVGMSRDAVKFGRMSKKQRDSLYAEVQKHRMQQQQRDHQQQPGEAEPLTPTYNISANGLTELHDDLSNYIDGHTPEGSKADSAVSSFYLDIQPSPDQSGLDINGIKPEPICDYTPASGFFPYCSFTNGETSPTVSMAELEHLAQNISKSHLETCQYLREELQQITWQTFLQEEIENYQNKQREVMWQLCAIKITEAIQYVVEFAKRIDGFMELCQNDQIVLLKAGSLEVVFIRMCRAFDSQNNTVYFDGKYASPDVFKSLGCEDFISFVFEFGKSLCSMHLTEDEIALFSAFVLMSADRSWLQEKVKIEKLQQKIQLALQHVLQKNHREDGILTKLICKVSTLRALCGRHTEKLMAFKAIYPDIVRLHFPPLYKELFTSEFEPAMQIDG

SEQ ID NO: 9 - DNA, 3646 bp, Homo sapiens
ctgcctttggagaaagtggagtgtggcgcttggttgtcgttatttcttcggactgcttcgcggtgcacggattcagcttctgcccagtggggctttcagctgtttgcgcgtctctctgtccccctcccctccccccggcacacctctgtctacgatgaggaaaggtctgcgggcgacagcggcccgctgcggactgggactgggatacttgctgcaaatgctcgtgctacctgccctggccctgctcagcgccagcggcactggctccgccgcccaagatgatgacttttttcatgaactcccagaaacttttccttctgatccacctgagcctctgccacatttccttattgagcctgaagaagcttatattgtgaagaataagcctgtgaacctgtactgtaaagcaagccctgccacccagatctatttcaagtgtaatagtgaatgggttcatcagaaggaccacatagtagatgaaagagtagatgaaacttccggtctcattgtccgggaagtgagcattgagatttcgcgccagcaagtggaagaactctttggacctgaagattactggtgccagtgtgtggcctggagctccgcgggtaccacaaagagccggaaggcgtatgtgcgcattgcatatctacggaagacatttgagcaggaacccctaggaaaggaagtgtctttggaacaggaagtcttactccagtgtcgaccacctgaagggatcccagtggctgaggtggaatggttgaaaaatgaagacataattgatcccgttgaagatcggaatttttatattactattgatcacaacctcatcataaagcaggcccgactctctgatactgcaaattacacctgtgttgccaaaaacattgttgccaagaggaaaagtacaactgccactgtcatagtctatgtcaacggtggctggtccacctggacggagtggtctgtgtgtaacagccgctgtggacgagggtatcagaaacgtacaaggacttgtaccaacccggcaccactcaatgggggtgccttctgtgaagggcagagtgtgcagaaaatagcctgtactacgttatgcccagtggatggcaggtggacgccatggagcaagtggtctacttgtggaactgagtgcacccactggcgcaggagggagtgcacggcgccagcccccaagaatggaggcaaggactgcgacggcctcgtcttgcaatccaagaactgcactgatgggctttgcatgcagactgctcctgattcagatgatgttgctctctatgttgggattgtgatagcagtgatcgtttgcctggcgatctctgtagttgtggccttgtttgtgtatcggaagaatcatcgtgactttgagtcagatattattgactcttcggcactcaatgggggctttcarcctgtgaacatcaaggcagcaagacaagatctgctggctgtacccccagacctcacgtcagctgcagccatgtacagaggacctgtctatgccctgcatgacgtctcagacaaaatcccaatgaccaactctccaattctggatccactgcccaacctgaaaatcaaagtgtacaacacctcaggtgctgtctccccccaagatgacctctctgagtttacgtccaagctgtcccctcagatgacccagtcgttgttggagaatgaagccctcagcctgaagaaccagagtctagcaaggcagactgatccatcctgtaccgcatttggcagcttcaactcgctgggaggtcaccttattgttcccaattcaggagtcagcttgctgattcccgctggggccattccccaagggagagtctacgaaatgtatgtgactgtacacaggaaagaaactatgaggccacccatggatgactctcagacacttttgacccctgtggtgagctgtgggcccccaggagctctgctcacccgccccgtcgtcctcactatgcatcactgcgcagaccccaataccgaggactggaaaatactgctcaagaaccaggcagcacagggacagtgggaggatgtggtggtggtcggggaggaaaacttcaccaccccctgctacattaagctggatgcagaggcctgccacatcctcacagagaacctcagcacctacgccctggtaggacattccaccaccaaagcggctgcaaagcgcctcaagctggccatctttgggcccctgtgctgctcctcgctggagtacagcatccgagtctactgtctggatgacacccaggatgccctgaaggaaattttacatcttgagagacagacgggaggacagctcctagaagaacctaaggctcttcattttaaaggcagcacccacaacctgcgcctgtcaattcacgatatcgcccattccctctggaagagcaaattgctggctaaatatcaggaaattccattttaccatgtttggagtggatctcaaagaaacctgcactgcaccttcactctggaaagatttagcctgaacacagtggagctggtttgcaaactctgtgtgcggcaggtggaaggagaagggcagatcttccagctcaactgcaccgtgtcagaggaacctactggcatcgatttgccgctgctggatcctgcgaacaccatcaccacggtcacggggcccagtgctttcagcatccctctccctatccggcagaagctctgtagcagcctggatgccccccagacgagaggccatgactggaggatgctggcccataagctgaacctggacaggtacttgaattactttgccaccaaatccagcccaactggcgtaatcctggatctttgggaagcacagaacttcccagatggaaacctgagcatgctggcagctgtcttggaagaaatgggaagacatgaaacggtggtgtccttagcagcagaagggcagtattaaccaccatgctggaaggggaaatgaaggacaaaaatgcacagggagtctgtggccgtccaggtgaatcacagctgaggaggaaatccagatgagaccaatgcacttcacaggcaagaatgcagcaggagccagaaggaaaacagatacaactgcccatgtacatgcccactttactcggagatcatcacgggagttaagaaaaattgtgtaaatttgtaccttgaatttagctatcaacctaattttcctcttagttgggctgtatgctgtgtggtacaggatcttacagtttcctaggaaacgctttttattgctatccagatatatggataaactttcttaacaaacccaatttctacaaatgttgtttacatcaaattggacagggatgcagacactgtccatggctcgttctatttttgttcaaatcatttgaagttgaagctgtggacggtttgttgtgtctatttcagattagtaatttacagagaaatcacagacttttgctacaaatcgtgtgcatcaagtgtctcagataatcctcccatcagtgttctgtttctagaacttgtagaaccagtgttactgtttgtatcagggaagtggagaatctaagtgtaaaaaagaaataactaagactcctattccttggagggacccttctggtgccctttgggaataaagctgtagcactgc

SEQ ID NO: 10 - protein, 931 aa, Homo sapiens [sequence omitted]

SEQ ID NO: 11 - DNA, 6277 bp, Homo sapiens
ataaagcacatttagcattgttgtgttttaataactgcaaatgttctaaatgtgaataagagcatataattaagattttttgaataaactcctaaaattgaaatgtttgtgttgtctttatatcattaccctaatgcccatttttggttgaggtatttgacagccagaataatggctccccaaggatgtccatggttttggaatttaagaatatgttacattacatggcaaatctgtagaaattaaagtagcagatggatttaaagttgctaatctgctgactttaagagaggtagattttcttggattattccattgggtccaatattatcacaataatttctaaatgtggaggaaattagaagaagtgagtgctttgatacgagaagtacttgaaccatacttgctgggtgtgaagatgggggaaggggccactagccaaagaatgtaggtgtcctctggaagctggaaacagctagaaaacatgctttctgtagaggctgcagaaaggagctcagctctgggatcattctcagttccttaaggctgccctctggtcctagccttggctagcctaggccttggccttctcatagaatcacaatttattttttaaaaagtcatcaagagaattaatctctcaagcttgccatgatcatgtcttatgtaatctaatcaaggaaatggaatatcccctcatatccataagccctggccacactcaagagaaaaacatatacatggcaaatatagcagagaacaagaaatttggaggccatcttggaattctgtctacaacactcctggtattccctctgtgtgtttgtctttgtctctctccacatatatatacgtacatacacatccatgcacatatgtatttgtattacttttttcaatgtagacatatcagcttcttataatatttgatttctttgtttttccactagttcagtcatatttcgctgttttagctaccagatcccatctgatacatgaaaacaacatgaagaagataaagaaagtttctctatgcatcttttctctcgtgacattcaattgcatactcaccatcacccaaacagcaaaattattgactgacacttcaaataatctcttctaatacttatttacattaaaaatatttttctttatgagggtgaagtagaaattaattcacagctctgtactctggacaaagaaaaaactgtaacaaaaaaatcttcacactgatctaacactttatattctcatgagtcaagtgattaattaaattccaatgctcctctgaaggaggttgggcatttctataaattaaaaaatatcgccgggcatggtggctcacgtctgtaatcccagcactttgggaagccaaggcaggcagatcacgaggtcaggagatcgagaccatcctggcttacacggtgaaaccctgtctctactaaaaatacaaaaaaaaaaaaagttaggtgggcgtagtggcgggcgcctgtagtcccagctactcgggaggctgaggcagaagaatggcgtgaacccaggaggcggagcttgcagtgagccaagatcgtgccactgcactccagcctgggtgacagagcgagactccatctcaaaaaaaaaaaaaaaattataactaaagttttgaattaaatcattctgattgtccaggaatgcacacccttaaatatatgtaataaaaaattaaagatttcttatagaaaaacttattgaaaaagtccctgaaagataacaaatgatattataaactatgtatttctgtagtaaatttcccaaaaatacataattttatgagcaaaatggtgtggaatatataaaatattgtatgtatgtgtgtgtatataagctttttaagtgaagttaagaaacatgaatgaggacctatgaagcattatttctgtgcacgtaccaatatcctgttatttccagtgcccaaccctaagatctctgactctaccacattaacttcagactaaaaactaacccttttaaacacaccgtaaatgatttttttttgttaaatgcaacttaaaaatttcaaaatatataatagtaaaaatgatgtttaatatattgtgttgaaatgttaaagaataaactgtagtttgaatgcatctgaatattttgtaggcatgcaacagctcttagtaagaaaccattccatctttctgagagaaagttgtatattaactgaagagaggcatcttgttttgatctggaggcacaagaaatcttaaagtgtgaatcccattaggttataagataaaaaaaaatcctatcagagaataggaaaataaaaaccactagtaatctcattactgcctcttcaaatactactgctatataggtgaacagcttcccctctagtctttcctctttcttcccaaaatagccgacgagtggatcaagcatgtctagtgggatataagcacaaagtatgcttattcttgttttctcctctttttatttgttctgattattaatttttcccctagtcatagttctttttttggatgaaatttagggcaagtgttcaggcttacctagttacttcagatgatgacatctcctctaaacagtaattaatattttcagtccatggtaatattcaacattttaagctcatttaaaatactaactatgccccctgatatggtttgactctgtgtctacacccaaatctcatgtcaaattgtaatccccacttattgaaggtggggactggtgggaggtgattggatcatagtcgggggttccagtggtttagcgccatcctccctagggctgtcttgtgatagagttctcaccagatccgatgagatctgatggtttcaaagtgtgtagcagtctcagtctctctgtctgtctgtctgtctctctctctgtctctcaatcccctgctccaccatggtaagatgtgcttacttcccctttatgttttgacacgattgtaagtttcctgaggcctcccagccatgcttctgtacagcctgaggaactgtgagtcaattaaatgtcttttcttcataaactacccagtctcaggtagttctttatagcagcatgaaaacagactaatacaccccctttctcagcctgtgcttgtggtaaggctagccaatgcaagcagaaagaggggtgctgtctgctgtttctccaattggcatctgtgccccttcttcatcttgtcagttatagtgggaggcccaagacttacttttggcctcagacacagtcaaggatcatcctgatgcaaaaaacaaaatcacctttgaacctcttcctccacaccatcttccactttggttttccgctatgcctttgtactggggacaaagactatccacgaatccttactttaaaaactgaggcagtggctctgaatcattcccccttggacacccccacacatctgaacaccctcgtgtcccaacaccacatctgctgaagttgactggacctgaaatgctggaggctcagtgttttcggaaactgcggagagttctcctgccacaattctcttgttgtccgtcaaaagcaattacatgtttcagatgatgaaaagcactgtcatgaaaagctggcacagcagccaaggagacagatgtattaaaccacacggcttctcagaactcccacttacgttggagctcagcacagagatgagcatggtgatatccccccgtctaacatcctcccatttggatttaatgcatgaggctcgtttctgatgaggtgccaaattctttcggtggacaaacacaaagttgcagaattcagtgatcatacaagtccatgtcctcaaacgaaaataataatactataaatgcaaagatgctaccataaaaaatacagtaggtggacttttgtgagtcaacataaaaaaatccagcatttctaactggtagcatgaggagtaccagtctgcaggatgttaacgcactgataggctatggacaatatattccacacttacggaagtggggaaactataggtcagacaaagctacaggtacttctttctttcttttttctttttttctttctgagatggagtctccctctgtcgcctaggctggagtgcaatggcatgatctcagctcactgcaacctccgcctcccgggttcaagtgattctcctgcctcagcctcctgagtcgctgggattataggcgcacaccaccaaccacccggctaatttttcatatttttagtagaaacgaggtttcactgtgttagccagaatggtctcgaactcctgacctaggtgatctgcctgcctcagcctcccaaagtgctgggattacaggtgtgagccacggcactcggcccaagtacttcttaactgcaggacttctcagaggcttttaatatggtaatgagtgctgtgcagtgagatgactcaaatcaatatatagccaacacgtttgatatgaccacagaacccatttccctgaacactttttatcacctcctagagcaggggtcagaaaactactgcccatgtggtctgatctggccactgcttgttttcgtaaattttcactgcccacagccaacctcatttgtgtacatgccgtccatggctgctttcacactgcagccgcagcgatgagtcatgagacacgcacccgtggctcacaaagtctaaaatatttactctttggccctttacggaaaacgtgtacaaactttgtcctctgaaatatagtttgggaacctacttcactcatgatatcaataattctaaaaaatttcaaacaatttttaaagtcaacattgagaagttttccatggttagaatttaataattacagtcaataactctcttaaagcatcgtgacaataaagtagaaggacactgtgtaattctatatggtctctgtcttcagggagctcccaagctagtttgtgaaaaagagaactcaaggcacaaacaactggggggcaagagatgttctaaacactaagaggaggcaatatggcatagtagaaagaatgcaagcatgtgtaagagtaaaacatacacagggcgcagagtcacaagagtcaggggttggtcttggctgtgataccttctcctgcgataagagtaagacctgctttattgagcacttactatgtgccaggcaccatcccagttgctttccatgaaggattgctctttaattcttatcataatgaataggtactattattatcccagtttatgggtgaggaaaccaagtcttaggttgactaacttattaaaataaagcacatacctagtaggtggtgaacaagagccaaaacaagtctgttggtcagctaaccaccacgttatagtggctgctgcttaggggcagcatatatcctctgcatttccccagaggtgacagaagtagtggagagggtagtgctctctaggctctgacacacagcccatgtttgctgactcaacaaccacacaacgtaatgctatggcacctcaggtggcagggagggtatcgtgggctggactgatcttcagaaggctgcatctgagcagggtgtggaaggatggcagaaggaagaggggagtgaggcactcagggtagacagaaggtgatactgccacatcctccactcagaaggaatctggaaggttttgacaaatatgtacattttggagtgggaacagcagaaagtaggggctggctacctactttccactacccctatccccactccccctagggggtgaggcagagataagcacgctttgtggggcacactgatccacaaacttgcaagcctgagctccactcagcagcagcctgggcaggatgaatggtggtgtgcaggtgtttgcggcatgcatagcttctctttctcctagagtcaatccagtgatcccctgcgaaaagaatcctccacttgacctgaatgctctgtattggcaccttctggacaggaatcctatcgggtatttcatcaaagcatcccacagcagttttaagtgtggctccatgagagtaagacaccaggacagctgtgccacctagtgatgcttgcctgtactgtctgcatcccagagagtgggagaggcttcggaaaggactggtatttgctatttttggccaacacttctacatgcaaaggaatctgaagtctccgcctgggcttcaaaacatttcttgtggcctcactgaccattcccgtgagcctcaggggacatgagtgtcttcaagctaagagggagcagtccaactctgcagcacgaattaccaaggaacat

SEQ ID NO: 12 - DNA, 1829 bp, Homo sapiens
ttttgccttggttttcccagctattaaaagggagatatcatcattcattcaacaagcatttcttgcggacccattttccacgacagaaccatccaaaccaagacacttcttcctattgtggcaaaaatgagcaacgttaaaactccccagaatgcagaggtgactcacgtgtttatgtaagtaagcatttagatatggctttggaaaagaaaacacacacctaaaagaggctaccagcatttgaataaagtgtgtaacctgggtgttcaaaactgtacaacagtttctgtgtactttaagtcactatccttcatacttcttccctttaaattctatttgaaactaaataatatcaacaatactttaagtaagattatgctggtgttactgatgaaattatatgcaggtctgcttggcctgttctcatgcgtaagcctttgataacttgttcctaggttatccagttctgcttacctgagtacccactttatcccagaaacgagacctgaacactgtgtcctggtttcatctttccagtgagccagccttctgaggcattagcggctcagcaagaccatggaggtgagagaggcacaggcacactccagatgcagggtctctgccctctgtcaagagtcacacggcagagaggacaagactcctgtcccgggggaactgtttttccttttgccccacaccccagatatcttcgtgacagcccctgccctgaaagcccggtgagcgtcatggcggagaagccagcctgtttccgattattctgcctgacagggactatctgctgctgtgtgcagttgtagaaatgtcataagctcaggactcaggacactttgacgtcgagtccaacaggacccatagagtccccctgaacaattctcatctgtgtgggaagcctaaggggtggttttaagtaggggctggcaggagagtgaggtctgggctgcttctttacggctcttcaagtgccggccatttcctgggattcccaggagtaagtcttgactcccatgaatcccactgacccctaagtggcatgggaaaggagaggacaaaaagaaaggagttagacatttgactaatactgttcagagttgaaatgcgctctttgagcaggtaggcatttaaagctaacttttcctcatgggatacacttgtgggttctttggagactgatctgcgtgaccccaatggctcaacgagaatcttctcctacaattcccttgactaggtaatgccctcactctcctgctatcttcttctccctggttctcagtgtttcactgccacataaactctctctctcactgttggggtcccattgcccctctgagtggaattcctgggcaaaagattcaatgcaagctcaccaggctatctcacattgggcctacatctggtctcctggaaatatgcccatatcctttgtctggacaatcctgagacagacccagcccaatgcagaagtgcctattcttctctcagcagcagattaacttgtctgggagttactcatcctcagacttctcaagctcattccgacacctgtccacctaatgcagcaactgcacgtgacacaataaacacttaatgcaagtcacaaatgggagtcacatatataatattattagtagccacgctagaaaaatgagacagaaacagataaagttaatttcaatttcagtaacatttcatttaatctaatctgtcaaaaatactattgtctcaatatataatcaacaaaactgttaatgaaatacattacagtttttttgtattaagtctttgaaatggagtgtgtattttagacttaa

SEQ ID NO: 13 - DNA, 2612 bp, Homo sapiens
atgaggaaaggtctgcgggcgacagcggcccgctgcggactgggactgggatacttgctgcaaatgctcgtgctacctgccctggccctgctcagcgccagcggcactggctccgccgcccaagatgatgacttttttcatgaactcccagaaacttttccttctgatccacctgagcctctgccacatttccttattgagcctgaagaagcttatattgtgaagaataagcctgtgaacctgtactgtaaagcaagccctgccacccagatctatttcaagtgtaatagtgaatgggttcatcagaaggaccacatagtagatgaaagagtagatgaaacttccggtctcattgtccgggaagtgagcattgagatttcgcgccagcaagtggaagaactctttggacctgaagattactggtgccagtgtgtggcctggagctccgcgggtaccacaaagagccggaaggcgtatgtgcgcattgcatatctacggaagacatttgagcaggaacccctaggaaaggaagtgtctttggaacaggaagtcttactccagtgtcgaccacctgaagggatcccagtggctgaggtggaatggttgaaaaatgaagacataattgatcccgttgaagatcggaatttttatattactattgatcacaacctcatcataaagcaggcccgactctctgatactgcaaattacacctgtgttgccaaaaacattgttgccaagaggaaaagtacaactgccactgtcatagtctatgtcaacggtggctggtccacctggacggagtggtctgtgtgtaacagccgctgtggacgagggtatcagaaacgtacaaggacttgtaccaacccggcaccactcaatgggggtgccttctgtgaagggcagagtgtgcagaaaatagcctgtactacgttatgcccagtggatggcaggtggacgccatggagcaagtggtctacttgtggaactgagtgcacccactggcgcaggagggagtgcacggcgccagcccccaagaatggaggcaaggactgcgacggcctcgtcttgcaatccaagaactgcactgatgggctttgcatgcagactgctcctgattcagatgatgttgctctctatgttgggattgtgatagcagtgatcgtttgcctggcgatctctgtagttgtggccttgtttgtgtatcggaagaatcatcgtgactttgagtcagatattattgactcttcggcactcaatgggggctttcagcctgtgaacatcaaggcagcaagacaagatctgctggctgtacccccagacctcacgtcagctgcagccatgtacagaggacctgtctatgccctgcatgacgtctcagacaaaatcccaatgaccaactctccaattctggatccactgcccaacctgaaaatcaaagtgtacaacacctcaggtgctgtcaccccccaagatgacctctctgagtttacgtccaagctgtcccctcagatgacccagtcgttgttggagaatgaagccctcagcctgaagaaccagagtctagcaaggcagactgatccatcctgtaccgcatttggcagcttcaactcactgggaggtcaccttattgttcccaattcaggagtcagcttgctgattcccgctggggccattccccaagggagagtctacgaaatgtatgtgactgtacacaggaaagaaactatgaggccacccatggatgactctcagacacttttgacccctgtggtgagctgtgggcccccaggagctctgctcacccgccccgtcgtcctcactatgcatcactgcgcagaccccaataccgaggactggaaaatactgctcctcgctggagtacagcatccgagtctactgtctggatgacacccaggatgccctgaaggaaattttacatcttgagagacagacgggaggacagctcctagaagaacctaaggctcttcattttaaaggcagcacccacaacctgcgcctgtcaattcacgatatcgcccattccctctggaagagcaaattgctggctaaatatcaggaaattccattttaccatgtttggagtggatctcaaagaaacctgcactgcaccttcactctggaaagatttagcctgaacacagtggagctggtttgcaaactctgtgtgcggcaggtggaaggagaagggcagatcttccagctcaactgcaccgtgtcagaggaacctactggcatcgatttgccgctgctggatcctgcgaacaccatcaccacggtcacggggcccagtgctttcagcatccctctccctatccggcagaagctctgtagcagcctggatgccccccagacgagaggccatgactggaggatgctggcccataagctgaacctggacaggtacttgaattactttgccaccaaatccagcccaactggcgtaatcctggatctttgggaagcacagaacttcccagatggaaacctgagcatgctggcagctgtcttggaagaaatgggaagacatgaaacggtggtgtccttagcagcagaagggcagtattaaccaccatgctggaaggggaaat

SEQ ID NO: 14 - protein, 636 aa, Homo sapiens
MRKGLRATAARCGLGLGYLLQMLVLPALALLSASGTGSAAQDDDFFHELPETFPSDPPEPLPHFLIEPEEAYIVKNKPVNLYCKASPATQIYFKCNSEWVHQKDHIVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPVEDRNFYITIDHNLIIKQARLSDTANYTCVAKNIVAKRKSTTATVIVYVNGGWSTWTEWSVCNSRCGRGYQKRTRTCTNPAPLNGGAFCEGQSVQKIACTTLCPVDGRWTPWSKWSTCGTECTHWRRRECTAPAPKNGGKDCDGLVLQSKNCTDGLCMQTAPDSDDVALYVGIVIAVIVCLAISVVVALFVYRKNHRDFESDIIDSSALNGGFQPVNIKAARQDLLAVPPDLTSAAAMYRGPVYALHDVSDKIPMTNSPILDPLPNLKIKVYNTSGAVTPQDDLSEFTSKLSPQMTQSLLENEALSLKNQSLARQTDPSCTAFGSFNSLGGHLIVPNSGVSLLIPAGAIPQGRVYEMYVTVHRKETMRPPMDDSQTLLTPVVSCGPPGALLTRPVVLTMHHCADPNTEDWKILLLAGVQHPSLLSG

SEQ ID NO: 15 - DNA, 28 bp, Artificial Sequence
  Forward Primer MF402L11I
atggagacttcgctcactggtcctcttt

SEQ ID NO: 16 - DNA, 28 bp, Artificial Sequence
  Reverse  Primer MF402L11I
cctggaggcacataaaccctagacatcc

SEQ ID NO: 17 - DNA, 21 bp, Artificial Sequence
  Forward Primer MF402L11II
acatcctgaggtaggtggtcc

SEQ ID NO: 18 - DNA, 21 bp, Artificial Sequence
  Reverse Primer MF402L11II
gggtctcactcattctgttgc

SEQ ID NO: 19 - DNA, 21 bp, Artificial Sequence
  Forward Primer MF402L11III
ctgtattttgactcaattgtg

SEQ ID NO: 20 - DNA, 21 bp, Artificial Sequence
  Reverse Primer MF402L11III
ccaaaagcctctccttattac

SEQ ID NO: 21 - DNA, 28 bp, Artificial Sequence
  Forward Primer MF402L11IV
gaatacagacacacagagacagcacctt

SEQ ID NO: 22 - DNA, 28 bp, Artificial Sequence
  Reverse Primer MF402L11IV
gttccttgctccagtgaaagacataatc

SEQ ID NO: 23 - DNA, 28 bp, Artificial Sequence
  Forward Primer MF402L11V
ctaacaccagttcagtggaacccattgc

SEQ ID NO: 24 - DNA, 28 bp, Artificial Sequence
  Reverse Primer MF402L11V
atttgatgttcatacagggttgggggac

SEQ ID NO: 25 - DNA, 28 bp, Artificial Sequence
  Forward Primer MF402L11VI
ctccacttggactctgatgacaccactg

SEQ ID NO: 26 - DNA, 27 bp, Artificial Sequence
  Reverse Primer MF402L11VI
catagttcactgcagcctcaacctcct

SEQ ID NO: 27 - DNA, 28 bp, Artificial Sequence
  Forward Primer MF90A19I
taattatgattagacagaccgtcctcca

SEQ ID NO: 28 - DNA, 28 bp, Artificial Sequence
  Reverse Primer MF90A19I
agtttactactcagcctttgccttcact

SEQ ID NO: 29 - DNA, 28 bp, Artificial Sequence
  Forward Primer MF90A19II
tctgccccacgtccaatctaggtaactt

SEQ ID NO: 30 - DNA, 28 bp, Artificial Sequence
  Reverse Primer MF90A19II
gatggtgtggaggaagaggttcaaaggt

SEQ ID NO: 31 - DNA, 28 bp, Artificial Sequence
  Forward Primer MF90A19III
aactgagaactggaaggtagctttggga

SEQ ID NO: 32 - DNA, 28 bp, Artificial Sequence
  Reverse Primer MF90A19III
acaagagcagtcagaactatttggccct

SEQ ID NO: 33 - DNA, 28 bp, Artificial Sequence
  Forward Primer MF90A19IV
atttgctggctccgacagtaagtattcc

SEQ ID NO: 34 - DNA, 28 bp, Artificial Sequence
  Reverse Primer MF90A19IV
atgcagtgatgcttaacaccctgtctct

SEQ ID NO: 35 - DNA, 28 bp, Artificial Sequence
  Forward Primer MF90A19V
cctgctctgtctgccttggaaaagaaat

SEQ ID NO: 36 - DNA, 28 bp, Artificial Sequence
  Reverse Primer MF90A19V
tcttggcccagacggtataagagtccat

SEQ ID NO: 37 - DNA, 28 bp, Artificial Sequence
  Forward Primer MF90A19VI
agtgcaccagtaagcatgccttccaata

SEQ ID NO: 38 - DNA, 28 bp, Artificial Sequence
  Reverse Primer MF90A19VI
aagctggcaaggagattctcaaaagtgg

SEQ ID NO: 39 - DNA, 28 bp, Artificial Sequence
  Forward Primer MF90A19VII
accacatctgctgaagttgactggacct

SEQ ID NO: 40 - DNA, 28 bp, Artificial Sequence
  Reverse Primer MF90A19VII
tctgggcaacatagtgaaaccctgtctc

SEQ ID NO: 41 - DNA, 28 bp, Artificial Sequence
  Forward Primer MF90A19VIII
cttgaacctaggagacggaggttgcagt

SEQ ID NO: 42 - DNA, 28 bp, Artificial Sequence
  Reverse Primer MF90A19VIII
aagcatatctctgaccctctggccattc

SEQ ID NO: 43 - DNA, 28 bp, Artificial Sequence
  Forward Primer MF90A19IX
gaagctgcaatcccatccttgtctcttt

SEQ ID NO: 44 - DNA, 28 bp, Artificial Sequence
  Reverse Primer MF90A19IX
tggtggtgcacacctgtaataccagcta

SEQ ID NO: 45 - DNA, 20 bp, Artificial Sequence
  Forward Primer C4P3F
aggatgtccatggttttgga

SEQ ID NO: 46 - DNA, 20 bp, Artificial Sequence
  Reverse Primer C4P3R
ctgtttccagcttccagagg

SEQ ID NO: 47 - DNA, 20 bp, Artificial Sequence
  Forward Primer C4P4F
catggtcccaaagctctgtt

SEQ ID NO: 48 - DNA, 20 bp, Artificial Sequence
  Reverse Primer C4P4R
tgacggaatttcagtgcaac

SEQ ID NO: 49 - DNA, 20 bp, Artificial Sequence
  Forward Primer C4P7F
tctgatacaggggatttggc

SEQ ID NO: 50 - DNA, 20 bp, Artificial Sequence
  Reverse Primer C4P7R
ttcaggtgggtgatgtgtgt

SEQ ID NO: 51 - DNA, 20 bp, Artificial Sequence
  Forward Primer C4P8F
aggaaagaggggtgctgtct

SEQ ID NO: 52 - DNA, 20 bp, Artificial Sequence
  Reverse Primer C4P8R
atagcaggagagtgagggca

SEQ ID NO: 53 - DNA, 20 bp, Artificial Sequence
  Forward Primer C4P9F
ggcccactttctggttcata

SEQ ID NO: 54 - DNA, 20 bp, Artificial Sequence
  Reverse Primer C4P9R
tcagccagaccagtgtgaaa

SEQ ID NO: 55 - DNA, 20 bp, Artificial Sequence
  Forward Primer C15P1F
ggtctctgccctctgtcaag

SEQ ID NO: 56 - DNA, 20 bp, Artificial Sequence
  Reverse Primer C15P1R
aggcttcccacacagatgag

SEQ ID NO: 57 - DNA, 20 bp, Artificial Sequence
  Forward Primer C15P2F
cactcttggcacccttaacc

SEQ ID NO: 58 - DNA, 20 bp, Artificial Sequence
  Reverse Primer C15P2R
tttgttctgtcctccccaac

SEQ ID NO: 59 - DNA, 20 bp, Artificial Sequence
  Forward Primer C15P5F
tttcatcaaagcatcccaca

SEQ ID NO: 60 - DNA, 20 bp, Artificial Sequence
  Reverse Primer C15P5R
tggtaattcgtgctgcagag

SEQ ID NO: 61 - DNA, 20 bp, Artificial Sequence
  Forward Primer C15P6F
cccaaacattccaacttcgt

SEQ ID NO: 62 - DNA, 20 bp, Artificial Sequence
  Reverse Primer C15P6R
tagctcaagcctgaacccat

SEQ ID NO: 63 - DNA, 26 bp, Artificial Sequence
  Forward Primer BPC4F
accctaatgcccatttttggttgagg

SEQ ID NO: 64 - DNA, 28 bp, Artificial Sequence
  Reverse Primer BPC15R
aaataccagtcctttccgaagcctctcc

SEQ ID NO: 65 - DNA, 28 bp, Artificial Sequence
  Forward Primer BPC15F
cgacagaaccatccaaaccaagacactt

SEQ ID NO: 66 - DNA, 28 bp, Artificial Sequence
  Reverse Primer BPC4R
gtgtttattgtgtcacgtgcagttgctg

SEQ ID NO: 67 - DNA, 27 bp, Artificial Sequence
  Forward Primer RORa1 5′ UTR
ctgaaaacagaagatagagggagtctc

SEQ ID NO: 68 - DNA, 20 bp, Artificial Sequence
  Reverse Primer UNC5c 3′UTR
atttccccttccagcatggt

SEQ ID NO: 69 - DNA, 27 bp, Artificial Sequence
  Forward Primer AP1
ccatcctaatacgactcactatagggc

SEQ ID NO: 70 - DNA, 22 bp, Artificial Sequence
  Reverse Primer
ctccacagatcttgcatggaat

SEQ ID NO: 71 - DNA, 23 bp, Artificial Sequence
  Forward Primer AP2
actcactatagggctcgagcggc

SEQ ID NO: 72 - DNA, 23 bp, Artificial Sequence
  Forward Primer
tctgtgtttcatacccaggaggt

SEQ ID NO: 73 - DNA, 23 bp, Artificial Sequence
  Reverse Primer
agaacacacacccatagactgca

SEQ ID NO: 74 - DNA, 19 bp, Artificial Sequence
  Forward Primer
cctgtaccgcatttggcag

SEQ ID NO: 75 - DNA, 20 bp, Artificial Sequence
  Reverse Primer
tgcaaaccagctccactgtg